(19) United States
(12) Patent Application Publication
(10) Pub. No.: US 2003/0097004 A1
(43) Pub. Date: May 22, 2003

(54) 3,4-Di-substituted cyclobutene-1,2-diones as CXC chemokine receptor antagonists

(75) Inventors: Arthur G. Taveras, Denville, NJ (US); Cynthia J. Aki, Livingston, NJ (US); Richard W. Bond, Union, NJ (US); Jianping Chao, Summit, NJ (US); Michael Dwyer, Scotch Plains, NJ (US); Johan A. Ferreira, Bensalem, PA (US); Jonathan A. Pachter, Maplewood, NJ (US); John J. Baldwin, Gwynedd Valley, PA (US); Bernd Kaiser, Plainsboro, NJ (US); Ge Li, ShangHai (CN); J. Robert Merritt, Ewing, NJ (US); Kingsley H. Nelson JR., Mebane, NC (US); Laura L. Rokosz, Union, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/062,006

(22) Filed: Feb. 1, 2002

Related U.S. Application Data
(60) Provisional application No. 60/265,951, filed on Feb. 2, 2001.

(51) Int. Cl. C07D 417/02; C07D 413/02

(52) U.S. Cl. 548/152; 548/217; 548/465; 564/306

(57) ABSTRACT

There are disclosed compounds of the formula

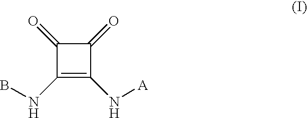

a prodrug thereof, or a pharmaceutically acceptable salt, solvate or isomer of said compound or of said prodrug, which are useful for the treatment of chemokine-mediated diseases such as acute and chronic inflammatory disorders and cancer.

### BACKGROUND OF THE INVENTION

This invention relates to novel substituted cyclobutenedione compounds, pharmaceutical compositions containing the compounds, and the use of the compounds and compositions in treating CXC-chemokine-mediated diseases.

Chemokines are chemotactic cytokines that are released by a wide variety of cells to attract macrophages, T-cells, eosinophils, basophils, neutrophils and endothelial cells to sites of inflammation and tumor growth. There are two main classes of chemokines, the CXC-chemokines and the CC-chemokines. The class depends on whether the first two cysteines are separated by a single amino acid (CXC-chemokines) or are adjacent (CC-chemokines). The CXC-chemokines include interleukin-8 (IL-8), neutrophil-activating protein-1 (NAP-1), neutrophil-activating protein-2 (NAP-2) GROα, GROβ, GROγ, ENA-78, IP-10, MIG and PF4. CC chemokines include RANTES, MIP-1α, MIP-2β, monocyte chemotactic protein-1 (MCP-1), MCP-2, MCP-3, GCP-2 and eotaxin. Individual members of the chemokine families are known to be bound by at least one chemokine receptor, with CXC-chemokines generally bound by members of the CXCR class of receptors, and CC-chemokines by members of the CCR class of receptors. For example, IL-8 is bound by the CXCR-1 and CXCR-2 receptors.

Since CXC-chemokines promote the accumulation and activation of neutrophils, these chemokines have been implicated in a wide range of acute and chronic inflammatory disorders including psoriasis and rheumatoid arthritis, Baggiolini et al., FEBS Lett. 307, 97 (1992); Miller et al., Crit. Rev. Immunol. 12, 17 (1992); Oppenheim et al., Annu. Fev. Immunol. 9, 617 (1991); Seitz et al., J. Clin. Invest. 87, 463 (1991); Miller et al., Am. Rev. Respir. Dis. 146,427 (1992); Donnely et al., Lancet 341,643(1993).

ELRCXC chemokines including IL-8, GROα, GROβ, GROγ, NAP-2, and ENA-78 (Strieter et al. 1995 JBC 270 p. 27348-57) have also been implicated in the induction of tumor angiogenesis (new blood vessel growth). All of these chemokines are believed to exert their actions by binding to the 7 transmembrane G-protein coupled receptor CXCR2 (also known as IL-8RB), while IL-8 also binds CXCR1 (also known as IL-8RA). Thus, their angiogenic activity is due to their binding to and activation of CXCR2, and possibly CXCR1 for IL-8, expressed on the surface of vascular endothelial cells (ECs) in surrounding vessels.

Many different types of tumors have been shown to produce ELRCXC chemokines and their production has been correlated with a more aggressive phenotype (Inoue et al. 2000 Clin Cancer Res 6 p. 2104-2119) and poor prognosis (Yoneda et. al. 1998 J Nat Cancer Inst 90 p. 447-454). Chemokines are potent chemotactic factors and the ELRCXC chemokines have been shown to induce EC chemotaxis. Thus, these chemokines probably induce chemotaxis of endothelial cells toward their site of production in the tumor. This may be a critical step in the induction of angiogenesis by the tumor. Inhibitors of CXCR2 or dual inhibitors of CXCR2 and CXCR1 will inhibit the angiogenic activity of the ELRCXC chemokines and therefore block the growth of the tumor. This anti-tumor activity has been demonstrated for antibodies to IL-8 (Arenberg et al. 1996 J Clin Invest 97 p. 2792-2802), ENA-78 (Arenberg et al. 1998 J Clin Invest 102 p. 465-72), and GROα (Haghnegahdar et al. J. Leukoc Biology 2000 67 p. 53-62).

Many tumor cells have also been shown to express CXCR2 and thus tumor cells may also stimulate their own growth when they secrete ELRCXC chemokines. Thus, along with decreasing angiogenesis, inhibitors of CXCR2 may directly inhibit the growth of tumor cells.

Hence, the CXC-chemokine receptors represent promising targets for the development of novel anti-inflammatory and anti-tumor agents.

There remains a need for compounds that are capable of modulating activity at CXC-chemokine receptors. For example, conditions associated with an increase in IL-8 production (which is responsible for chemotaxis of neutrophil and T-cell subsets into the inflammatory site and growth of tumors) would benefit by compounds that are inhibitors of IL-8 receptor binding. SUMMARY OF THE INVENTION

This invention provides novel compounds of Formula (I) represented by the structure:

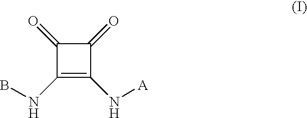

a prodrug thereof, or a pharmaceutically acceptable salt, solvate or isomer of said compound or of said prodrug;

wherein

A is an unsubstituted or substituted aryl or unsubstituted or substituted heteroaryl group;

B is

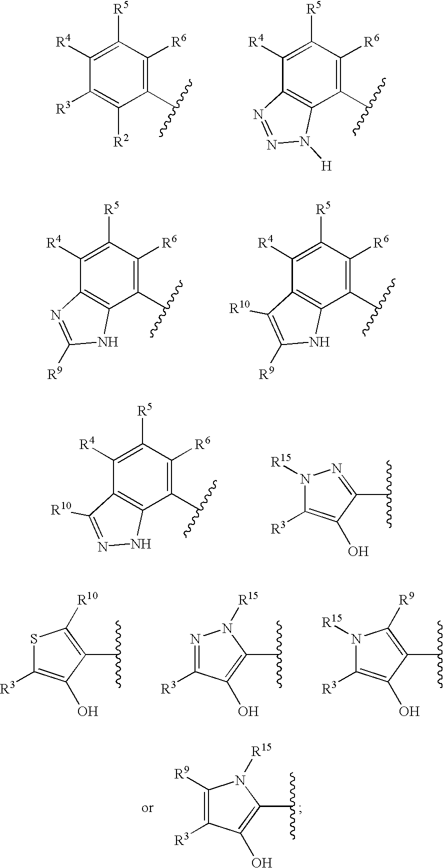

R² is hydrogen, OH, C(O)OH, SH, SO₂NR⁷R⁸, NHC(O)R⁷, NHSO₂NR⁷R⁸, NHSO₂R⁷, C(O)NR⁷R⁸, C(O)N R⁷OR⁸, OR¹³ or an unsubstituted or substituted heterocyclic acidic functional group;

R³ and R⁴ are the same or different and are independently hydrogen, halogen, alkoxy, OH, CF₃, QCF₃, NO₂, C(O)R⁷, C(O)OR⁷, C(O)NR⁷R⁸, SO₍ₜ₎NR⁷R⁸, SO₍ₜ₎R⁷, C(O)NR⁷OR⁸,

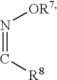

cyano, unsubstituted or substituted alkyl, unsubstituted or substituted aryl or unsubstituted or substituted heteroaryl;

R⁵ and R⁶ are the same or different and are independently hydrogen, halogen, alkyl, alkoxy, CF₃, OCF₃, NO₂, C(O)R⁷, C(O)OR⁷, C(O)NR⁷R⁸, SO₍ₜ₎NR⁷R⁸, C(O)NR⁷OR⁸, cyano, or an unsubstituted or substituted aryl or an unsubstituted or substituted heteroaryl group;

R⁷ and R⁸ are the same or different and are independently hydrogen, unsubstituted or substituted alkyl, unsubstituted or substituted aryl, unsubstituted or substituted alkylaryl, unsubstituted or substituted arylalkyl, unsubstituted or substituted cycloalkyl, carboxyalkyl, aminoalkyl, unsubstituted or substituted heteroaryl, unsubstituted or substituted heteroarylalkyl or unsubstituted or substituted heteroalkylaryl, or

R⁷, R⁸ and N in said NR⁷R⁸ and NR⁷OR⁸ can jointly form a 3 to 7 membered ring, said ring may further contain 1 to 3 additional heteroatoms on said ring as ring atoms, and said ring may be unsubstituted or substituted with one or more moieties which are the same or different, each moiety being independently selected from hydroxy, cyano, carboxyl, hydroxyalkyl, alkoxy, COR⁷R⁸ or aminoalkyl;

R⁹ and R¹⁰ are the same or different and are independently hydrogen, halogen, CF₃, OCF₃, NR⁷R⁸, NR⁷C(O)NR⁷R⁸, OH, C(O)OR⁷, SH, SO₍ₜ₎NR⁷R⁸, SO₂R⁷, NHC(O)R⁷, NHSO₂NR⁷R⁸, NHSO₂R⁷, C(O)NR⁷R⁸, C(O)NR⁷R⁸, OR¹³ or an unsubstituted or substituted heterocyclic acidic functional group;

1R³ is COR⁷;

R¹⁵ is hydrogen, OR¹³, or an unsubstituted or substituted aryl group, an unsubstituted or substituted heteroaryl group, an unsubstituted or substituted arylalkyl group, an unsubstituted or substituted cycloalkyl group or an unsubstituted or substituted alkyl group; and

t is 1 or 2.

Another aspect of the present invention is a pharmaceutical composition comprising the compound of formula (I) in combination or association with a pharmaceutically acceptable carrier or diluent.

Another aspect of the present invention is a method of treating an a-chemokine mediated disease in a mammal which comprises administering to a patient in need thereof of a therapeutically effective amount of the compound of formula (I), or a pharmaceutically acceptable salt or solvate thereof.

Another aspect of the present invention is a method of treating cancer, comprising administering to a patient in need thereof, concurrently or sequentially, a therapeutically effective amount of (a) a compound of formula (I), and (b) a microtubule affecting agent or antineoplastic agent or anti-angiogenesis agent or VEGF receptor kinase inhibitor or antibodies against the VEGF receptor or interferon, and/or c) radiation.

In preferred embodiments, a compound of formula (I) is combined with one of the following antineoplastic agents: gemcitabine, paclitaxel (Taxol®), 5-Fluorouracil (5-FU), cyclophosphamide (Cytoxan®), temozolomide, taxotere or Vincristine.

In another preferred embodiment, the present invention provides a method of treating cancer, comprising administering, concurrently or sequentially, an effective amount of (a) a compound of formula (I), and (b) a microtubule affecting agent (e.g., paclitaxel).

### DESCRIPTION OF THE PREFERRED EMBODIMENTS

Except where stated otherwise, the following definitions apply throughout the present specification and claims. Additionally, all technical and scientific terms used herein have the same meaning as is commonly understood by one of skill in the art to which this invention belongs. These definitions apply regardless of whether a term is used by itself or in combination with other terms. Hence the definition of “alkyl” applies to “alkyl” as well as to the “alkyl” portions of “alkoxy”, etc.

When any variable (e.g., aryl, R²) occurs more than one time in any constituent, its definition on each occurrence is independent of its definition at every other occurrence. Also, combinations of substituents and/or variables are permissible only if such combinations result in stable compounds.

The term “substituted” in the phrase “unsubstituted or substituted” refers to optional substitution with one or more moieties which are the same or different, each being independently selected from the group consisting of, halogen, hydroxy, cyano, nitro, alkyl, alkoxy, aryl, cycloalkyl, COOalkyl, COOaryl, carboxamide, sulfhydryl, arylalkyl, alkylaryl, amino, alkylamino, dialkylamino, alkylsulfonyl, arylsulfonyl, arylsulfonamido, alkylsulfonamido, heteroaryl, carboxyl, carboxyalkyl, heteroarylalkyl, heteroalkylaryl, and aryloxy. The term “substituted” also refers to substituting with a methylenedioxy group on two adjacent ring carbons on an aromatic ring, or by fusing a carbocyclic or heterocyclic ring onto two adjacent carbons on an aromatic ring.

Alkyl represents a straight or branched saturated hydrocarbon chain having the designated number of carbon atoms. Where the number of carbon atoms is not specified, 1 to 6 carbons are intended. Representative examples of alkyl groups include methyl, ethyl, n-propyl, iso-propyl, n-butyl, sec-butyl, iso-butyl, t-butyl and the like.

The term “cycloalkyl” means a non-aromatic mono- or multicyclic ring system comprising 3 to 10 carbon atoms, preferably 5 to 10 carbon atoms. The cycloalkyl can be optionally substituted on the ring by replacing an available hydrogen on the ring by one or more substituents which may be the same or different. Non-limiting examples of monocyclic cycloalkyls include cyclopropyl, cyclopentyl, cycolhexyl and the like. Non-limiting examples of multicyclic cycloalkyl rings include 1-decalinyl, norbornyl, adamantyl and the like.

The term halogen or Halo is intended to include fluorine, chlorine, bromine or iodine.

Aryl refers to a mono- or bicyclic ring system having one or two aromatic rings including, but not limited to, phenyl, naphthyl, indenyl, tetrahydronaphthyl, indanyl, anthracenyl, fluorenyl and the like.

The term heterocycle or heterocyclic ring is defined by all non-aromatic, heterocyclic rings of 3-7 atoms containing 1-3 heteroatoms selected from N, O and S, such as oxirane, oxetane, tetrahydrofuran, tetrahydropyran, pyrrolidine, piperidine, piperazine, tetrahydropyridine, tetrahydropyrimidine, tetrahydrothiophene, tetrahydrothiopyran, morpholine, hydantoin, valerolactam, pyrrolidinone, and the like.

Heteroaryl refers to 5- or 10-membered single or benzofused aromatic rings consisting of 1 to 3 heteroatoms independently selected from the group consisting of —O—, —S, and —N═, provided that the rings do not possess adjacent oxygen and/or sulfur atoms. The heteroaryl group can be unsubstituted or substituted with one, two, or three substituents independently selected from lower alkyl, halo, cyano, nitro, haloalkyl, hydroxy, alkoxy, carboxy, carboxyalkyl, carboxamide, sulfhydryl, amino, alkylamino and dialkylamino.

The term heterocyclic acidic functional group is intended to include groups such as, pyrrole, imidazole, triazole, tetrazole, and the like. Such groups can be unsubstituted or substituted with one, two, or three substituents independently selected from lower alkyl, alkyl, cycloalkyl, halo, cyano, nitro, haloalkyl, hydroxy, alkoxy, carboxy, carboxyalkyl, carbamoylalkyl, COOH, COOalkyl, COOaryl, carboxamide, sulfhydryl, amino, alkylamino, aminoalkyl, alkylaminoalkyl, aminoalkoxy, dialkylamino, sulfonyl, sulfonamido, aryl, heterocyclylalkyl and heteroaryl.

N-oxides can form on a tertiary nitrogen present in an R substituent, or on ═N— in a heteroaryl ring substituent and are included in the compounds of formula I.

As used herein, the term “composition” is intended to encompass a product comprising the specified ingredients in the specified amounts, as well as any product which results, directly or indirectly, from combination of the specified ingredients in the specified amounts.

The term “prodrug,” as used herein, represents compounds which are rapidly transformed in vivo to the parent compound of the above formula, for example, by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

For compounds of the invention having at least one asymmetrical carbon atom, all isomers, including diastereomers, enantiomers and rotational isomers are contemplated as being part of this invention. The invention includes d and/isomers in both pure form and in admixture, including racemic mixtures. Isomers can be prepared using conventional techniques, or by separating isomers of a compound of formula I.

Compounds of formula I can exist in unsolvated and solvated forms, including hydrated forms. In general, the solvated forms, with pharmaceutically acceptable solvents such as water, ethanol and the like, are equivalent to the unsolvated forms for purposes of this invention.

A compound of formula I may form pharmaceutically acceptable salts with organic and inorganic acids or bases. Examples of suitable acids for salt formation are hydrochloric, sulfuric, phosphoric, acetic, citric, malonic, salicylic, malic, fumaric, succinic, ascorbic, maleic, methanesulfonic and other mineral and carboxylic acids well known to those skilled in the art. The salts are prepared by contacting the free base forms with a sufficient amount of the desired acid to produce a salt in the conventional manner. The free base forms may be regenerated by treating the salt with a suitable dilute aqueous base solution, such as dilute aqueous sodium hydroxide, lithium hydroxide, potassium hydroxide, calcium hydroxide, potassium carbonate, ammonia or sodium bicarbonate. The neutral forms differ from their respective salt forms somewhat in certain physical properties, such as solubility in polar solvents, but the salts are otherwise equivalent to their respective neutral forms for purposes of the invention.

In a preferred group of compounds of formula I, A is selected from the group consisting of

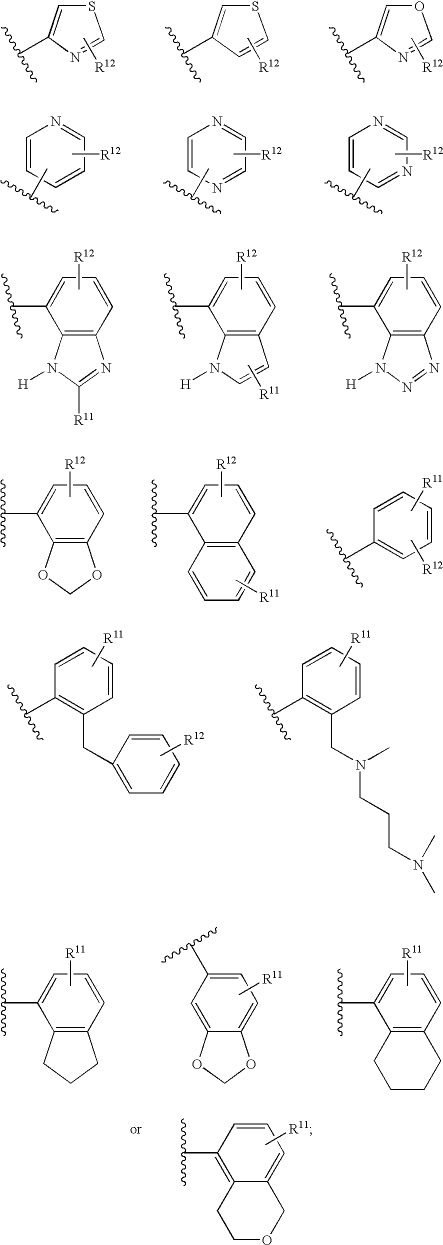

wherein

R¹¹ and R¹² are the same or different and are independently H, OH, halogen, cyano, CF₃, CF₃O, NR⁷R⁸, NR⁷C(O)NR⁷R⁸, C(O)NR⁷R⁸, CO₂R⁷, OR⁷, SO₍ₜ₎NR⁷R⁸, NR⁷SO₍ₜ₎R⁸, COR⁷, and substituted or unsubstituted aryl, substituted or unsubstituted alkyl, substituted or unsubstituted alkoxy, substituted or unsubstituted arylalkyl, substituted or unsubstituted heteroaryl, aryloxy, heteroarylalkyl, heteroarylalkoxy, heterocyclylalkyl, hydroxyalkyl, alkylaminoCOOalkyl, aminoalkoxy, alkoxyaminoalkyl and aminoalkyl; and

B is

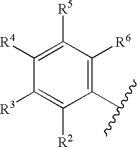

wherein

R² is selected from the group consisting of OH, NHC(O)R⁷ and NHSO₂R⁷;

R³ is selected from the group consisting of SO₂NR⁷R⁸, NO₂, CN, C(O)NR⁷R⁸ and SO₂R⁷;

R⁴ is selected from the group consisting of H, NO₂, CN and CF₃;

R⁵ is selected from the group consisting of H, CF₃, halogen and CN; and

R⁶ is selected from the group consisting of H and CF₃.

Compounds of formula (I) may be produced by processes known to those skilled in the art in the following reaction schemes and in the preparations and examples below.

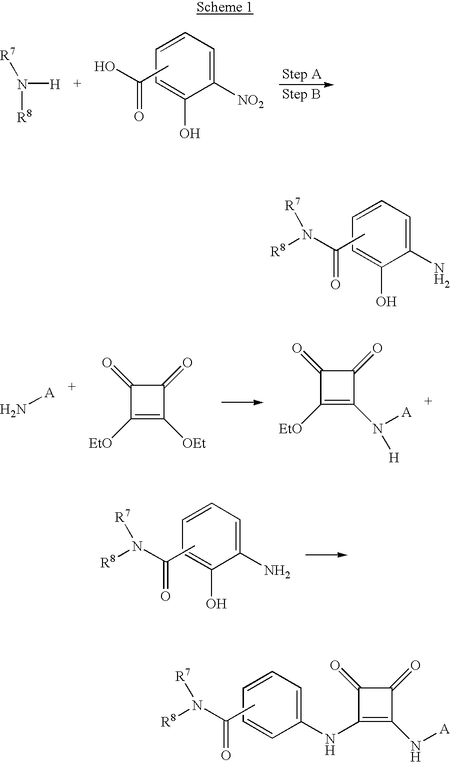

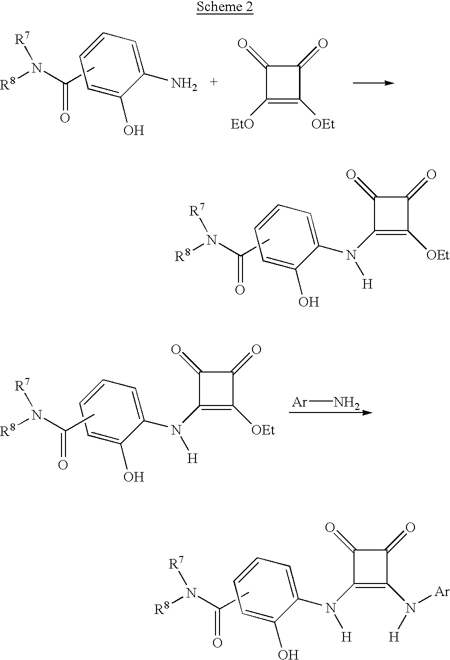

A general procedure for the preparation of compounds of formula I is as follows:

Scheme 1

An amine is condensed (Step A) with a nitrosalicylic acid under standard coupling conditions and the resulting nitrobenzamide is reduced (Step B) under hydrogen atmosphere in the presence of a suitable catalyst. The remaining partner required for the synthesis of the final target is prepared by condensing an aryl amine with the commercially available diethylsquarate to give the anilinoethoxysquarate product. Subsequent condensation of this intermediate with the aminobenzamide prepared earlier provides the desired chemokine antagonist (Scheme 1).

Scheme 2

Alternatively, the aminobenzamide of Scheme 1 is first condensed with commercially available diethylsquarate to give an alternate monoethoxy intermediate. Condensation of this intermediate with an aryl or heteroaryl amine gives the desired chemokine antagonist.

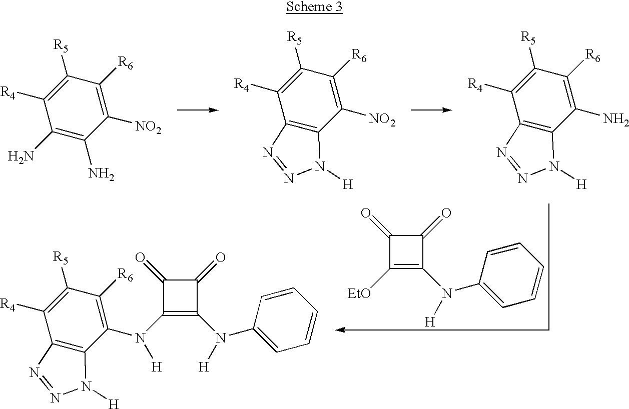

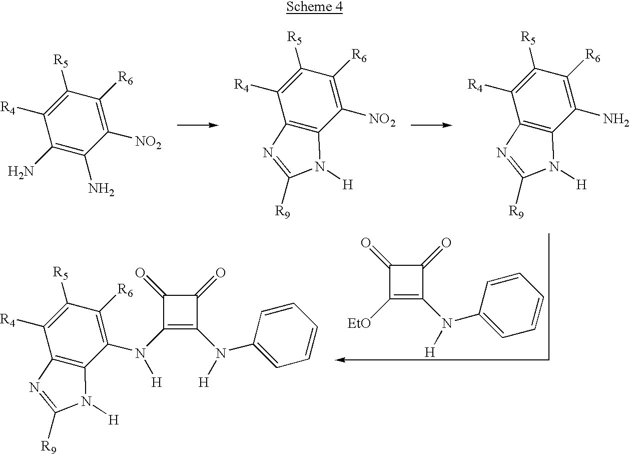

Scheme 3

Benztriazole compounds of Formula (I) are prepared by stirring nitrophenylenediamines with sodium nitrite in acetic acid at 60° C. to afford the nitrobenzotriazole intermediate (Scheme 3). Reduction of the nitro group in the presence of palladium catalyst and hydrogen atmosphere provided the amine compound. Subsequent condensation of this intermediate with the anilinoethoxysquarate prepared earlier (Scheme 1) provides the desired chemokine antagonist.

Scheme 4

Condensation of nitrophenylenediamines with anhydrides or activated acids at reflux (Scheme 4) affords benzimidazole intermediates which after reduction with hydrogen gas and palladium catalyst and condensation with the anilinoethoxysquarate previously prepared (Scheme 1) affords benzimidazole chemokine antagonists.

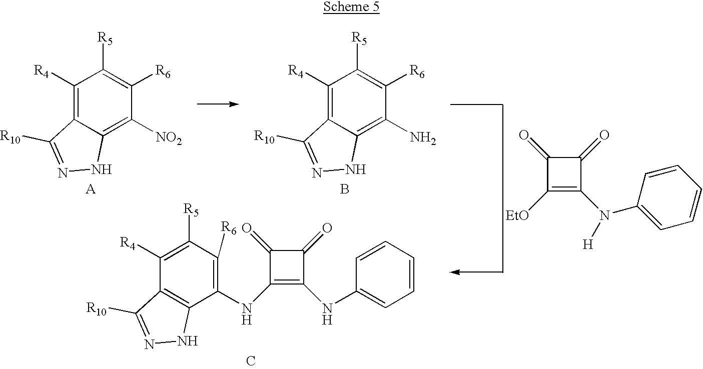

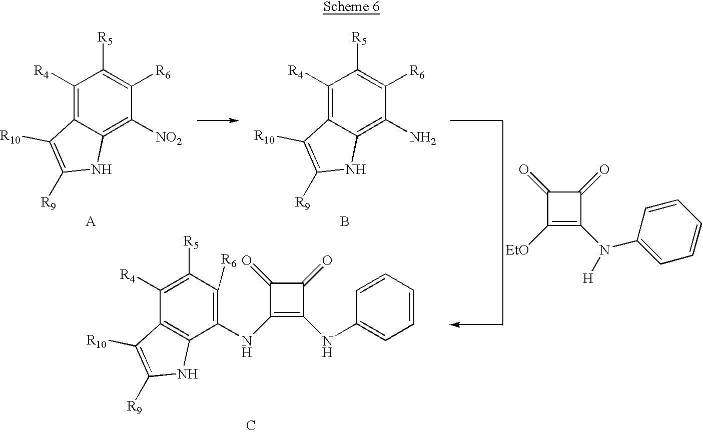

Scheme 5

Indazole structures of Formula (I) can be prepared according to Scheme 5 by reduction of nitroindazole A (J. Am. Chem Soc. 1943, 65, 1804-1805) to give aminoindazole B and subsequent condensation with the anilinoethoxysquarate prepared earlier (Scheme 1).

Scheme 6

Indole structures of Formula (I) can be prepared according to Scheme 6 by reduction of nitroindole A (J. Med. Chem. 1995, 38, 1942-1954) to give aminoindole B and subsequent condensation with the anilinoethoxysquarate prepared earlier (Scheme 1).

### BIOLOGICAL EXAMPLES

The compounds of the present invention are useful in the treatment of CXC-chemokine mediated conditions and diseases. This utility is manifested in their ability to inhibit IL-8 and GRO-α chemokine as demonstrated by the following in vitro assays.

Receptor Binding Assays:

CXCR1 SPA Assay

For each well of a 96 well plate, a reaction mixture of 10 μg hCXCR1-CHO overexpressing membranes (Biosignal) and 200 μg/well WGA-SPA beads (Amersham) in 100 μl was prepared in CXCR1 assay buffer (25 mM HEPES, pH 7.8, 2 mM CaCl₂, 1 mM MgCl₂, 125 mM NaCl, 0.1% BSA) (Sigma). A 0.4 nM stock of ligand, [125I]-IL-8 (NEN) was prepared in the CXCR1 assay buffer. 20× stock solutions of test compounds were prepared in DMSO (Sigma). A 6× stock solution of IL-8 (R&D) was prepared in CXCR2 assay buffer. The above solutions were added to a 96-well assay plate (PerkinElmer) as follows: 10 μl test compound or DMSO, 40 μl CXCR1 assay buffer or IL-8 stock, 100 μl of reaction mixture, 50 μl of ligand stock (Final [Ligand]=0.1 nM). The assay plates were shaken for 5 minutes on plate shaker, then incubated for 8 hours before cpm/well were determined in Microbeta Trilux counter (PerkinElmer). % Inhibition of Total binding-NSB (250 nM IL-8) was determined for IC50 values.

CXCR2 SPA Assay

For each well of a 96 well plate, a reaction mixture of 4 μg hCXCR2—CHO overexpressing membranes (Biosignal) and 200 μg/well WGA-SPA beads (Amersham) in 100 μl was prepared in CXCR2 assay buffer (25 mM HEPES, pH 7.4, 2 mM CaCl₂, 1 mM MgCl₂). A 0.4 nM stock of ligand, [125I]-IL-8 (NEN), was prepared in the CXCR2 assay buffer. 20× stock solutions of test compounds were prepared in DMSO (Sigma). A 6× stock solution of GRO-α (R&D) was prepared in CXCR2 assay buffer. The above solutions were added to a 96-well assay plate (PerkinElmer or Corning) as follows: 10 μl test compound or DMSO, 40 ul CXCR2 assay buffer or GRO-α a stock, 100 μl of reaction mixture, 50 μl of ligand stock (Final [Ligand]=0.1 nM). When 40× stock solutions of test compounds in DMSO were prepared, then the above protocol was used except instead 5 μl test compound or DMSO and 45 μl CXCR2 assay buffer were used. The assay plates were shaken for 5 minutes on a plate shaker, then incubated for 2-8 hours before cpm/well were determined in Microbeta Trilux counter (PerkinElmer). % Inhibition of total binding minus non-specific binding (250 nM Gro-α or 50 μM antagonist) was determined and IC50 values calculated.

Calcium Fluorescence Assay (FLIPR)

HEK 293 cells stably transfected with hCXCR2 and Gαι/q were plated at 10,000 cells per well in a Poly-D-Lysine Black/Clear plate (Becton Dickinson) and incubated 48 hours at 5% CO₂, 37° C. The cultures were then incubated with 4 mM fluo-4, AM (Molecular Probes) in Dye Loading Buffer (1% FBS, HBSS w. Ca & Mg, 20 mM HEPES (Cellgro), Probenicid (Sigma)) for 1 hour. The cultures were washed with wash buffer (HBSS w Ca, & Mg, 20 mM HEPES, Probenicid (2.5 mM)) three times, then 100 μl/well wash buffer was added.

During incubation, compounds were prepared as 4× stocks in 0.4% DMSO (Sigma) and wash buffer and added to their respective wells in the first addition plate. IL-8 or GRO-α (R&D Systems) concentrations were prepared 4× in wash buffer +0.1% BSA and added to their respective wells in second addition plate.

Culture plate and both addition plates were then placed in the FLIPR imaging system to determine change in calcium fluorescence upon addition of compound and then ligand. Briefly, 50 μl of compound solutions or DMSO solution was added to respective wells and change in calcium fluorescence measured by the FLIPR for 1 minute. After a 3 minute incubation within the instrument, 50 μl of ligand was then added and the change in calcium fluorescence measured by the FLIPR instrument for 1 minute. The area under each stimulation curve was determined and values used to determine % Stimulation by compound (agonist) and % Inhibition of Total Calcium response to ligand (0.3 nM IL-8 or GRO-α) for IC50 values of the test compounds.

Chemotaxis Assays for 293-CXCR2

A chemotaxis assay is setup using Fluorblok inserts (Falcon) for 293-CXCR2 cells (HEK-293 cells overexpressing human CXCR2). The standard protocol used at present is as follows:

1. Inserts are coated with collagen IV (2 ug/ml) for 2 hrs at 37° C.

2. The collagen is removed and inserts are allowed to air dry overnight.

3. Cells are labeled with 10 uM calcein AM (Molecular Probes) for 2 hrs. Labeling is done in complete media with 2% FBS.

4. Dilutions of compound are made in minimal media (0.1% BSA) and placed inside the insert which is positioned inside the well of a 24 well plate. Within the well is IL-8 at a concentration of 0.25 nM in minimal media. Cells are washed and resuspended in minimal media and placed inside the insert at a concentration of 50,000 cells per insert.

5. Plate is incubated for 2 hrs and inserts are removed and placed in a new 24 well. Fluorescence is detected at excitation=485 nM and emission=530 nM.

Cytotoxicity Assays

A cytotoxicity assay for CXCR2 compounds is conducted on 293-CXCR2 cells. Concentrations of compounds are tested for toxicity at high concentrations to determine if they may be used for further evaluation in binding and cell based assays. The protocol is as follows:

1.293-CXCR2 cells are plated overnight at a concentration of 5000 cells per well in complete media.

2. Dilutions of compound are made in minimal media w/0.1% BSA. Complete media is poured off and the dilutions of compound are added. Plates are incubated for 4, 24 and 48 hrs. Cells are labeled with 10 uM calcein AM for 15 minutes to determine cell viability. Detection method is the same as above.

Soft Agar Assay

10,000 SKMEL-5 cells/well are placed in a mixture of 1.2% agar and complete media with various dilutions of compound. Final concentration of agar is 0.6%. After 21 days viable cell colonies are stained with a solution of MTT (1 mg/ml in PBS). Plates are then scanned to determine colony number and size. IC₅₀ is determined by comparing total area vs. compound concentration.

For the compounds of this invention, a range of CXCR2 receptor binding activities from about 1 nM to about 10,000 nM was observed. Compounds of this invention preferably have a binding activity in the range of about 1 nM to 1,000 nM, more preferably about 1 to 500 nM, and most preferably about 1 nM to 100 nM.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for controlled release.

Formulations for oral use may also be presented as hard gelatin capsules wherein the active ingredients is mixed with an inert solid diluent, for example, calcium carbonate, calcium phosphate or kaolin, or a soft gelatin capsules where in the active ingredient is mixed with water or an oil medium, for example peanut oil, liquid paraffin or olive oil.

Aqueous suspensions contain the active material in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example, ethyl or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example, arachis oil, olive oil, sesame oil or coconut oil, or in mineral oil such as liquid paraffin. The oily suspensions may contain a thickening agent, for example, beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, e.g., sweetening, flavoring and coloring agents, may also be present.

The pharmaceutical compositions of the invention may also be in the form of an oil-in-water emulsions. The oily phase may be a vegetable oil, e.g., olive oil or arachis oil, or a mineral oil, e.g., liquid paraffin or mixtures of these. Suitable emulsifying agents may be naturally-occurring phosphatides, e.g., soy beans, lecithin, and esters or partial esters derived from fatty acids and hexitol anhydrides, for example, sorbitan monooleate, and condensation products of the said partial esters with ethylene oxide, e.g., polyoxyethylene sorbitan monooleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, e.g., as a solution in 1,3-butane diol. Among the acceptable vehicles and solvents that may be employed are water, Ringer's solution and isotonic sodium chloride solution. In addition, sterile fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of the invention may also be administered in the form of suppositories for rectal administration of the drug. The compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compound of The invention are employed. (For purposes of this application, topical application shall include mouthwashes and gargles.)

The compounds for the present invention can be administered in the intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethyleme glycols of various molecular weights and fatty acid esters of polyethylene glycol.

The dosage regimen utilizing the compounds of the present invention is selected in accordance with a variety of factors including type, species, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter, arrest or reverse the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug. Preferably, doses of the compound of structural The invention useful in the method of the present invention range from 0.01 to 1000 mg per adult human per day. Most preferably, dosages range from 0.1 to 500 mg/day. For oral administration, the compositions are preferably provided in the form of tablets containing 0.01 to 1000 milligrams of the active ingredient, particularly 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. An effective amount of the drug is ordinarily supplied at a dosage level of from about 0.0002 mg/kg to about 50 mg/kg of body weight per day. The range is more particularly from about 0.001 mg/kg to 1 mg/kg of body weight per day.

Advantageously, the active agent of the present invention may be administered in a single daily dose, or the total daily dosage may be administered in dividend doses of two, three or four time daily.

The amount of active ingredient that may be combined with the carrier materials to produce single dosage form will vary depending upon the host treated and the particular mode of administration.

It will be understood, however, that the specific dose level for any particular patient will depend upon a variety of factors including the age, body weight, general health, sex, diet, time of administration, route or administration, rate of excretion, drug combination and the severity of the particular disease undergoing therapy.

Another aspect of the invention is a method for treating cancer, comprising administering to a patient in need thereof, concurrently or sequentially, a therapeutically effective amount of (a) a compound of formula (I) and (b) an anti-cancer agent such as an antineoplastic agent, a microtubule affecting agent or an anti-angiogenesis agent. Additionally, the compounds of the invention can be co-administered with radiation therapy.

Classes of compounds that can be used as the anti-cancer chemotherapeutic agent (antineoplastic agent) include alkylating agents, antimetabolites, natural products and their derivatives, hormones, anti-hormones, anti-angiogenic agents and steroids (including synthetic analogs), and synthetics. Examples of compounds within these classes are given below.

Alkylating agents (including nitrogen mustards, ethylenimine derivatives, alkyl sulfonates, nitrosoureas and triazenes): Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Antimetabolites (including folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Natural products and their derivatives (including vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, paclitaxel (paclitaxel is commercially available as Taxol® and is described in more detail below in the subsection entitled “Microtubule Affecting Agents”), Mithramycin, Deoxyco-formycin, Mitomycin-C, L-Asparaginase, Interferons (especially IFN-α), Etoposide, and Teniposide.

Hormones and steroids (including synthetic analogs): 17α-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Zoladex.

Synthetics (including inorganic complexes such as platinum coordination complexes): Cisplatin, Carboplatin, Hydroxyurea, Amsacrine, Procarbazine, Mitotane, Mitoxantrone, Levamisole, and Hexamethylmelamine.

Anti-angiogenic agents include Marimastat, AG3340, Col-3, Neovastat, BMS-275291, Thalidomide, Squalamine, Endostatin, SU-5416, SU-6668, Interferon-alpha, Anti-VEGF antibody, EMD121974, CAI, Interleukin-12, IM862, Platelet Factor-4 Vitaxin, Angiostatin, Suramin, TNP470, PTK-787, ZD-6474, ZD-101, Bay 129566, CGS27023A, taxotere and Taxol.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, NJ 07645-1742, USA); the disclosure of which is incorporated herein by reference thereto.

As used herein, a microtubule affecting agent is a compound that interferes with cellular mitosis, i.e., having an anti-mitotic effect, by affecting microtubule formation and/or action. Such agents can be, for instance, microtubule stabilizing agents or agents which disrupt microtubule formation.

Microtubule affecting agents useful in the invention are well known to those of skill in the art and include, but are not limited to allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), Taxol® derivatives (e.g., derivatives (e.g., NSC 608832), thiocolchicine (NSC 361792), trityl cysteine (NSC 83265), vinblastine sulfate (NSC 49842), vincristine sulfate (NSC 67574), epothilone A, epothilone, and discodermolide (see Service, (1996) Science, 274:2009) estramustine, nocodazole, MAP4, and the like. Examples of such agents are also described in the scientific and patent literature, see, e.g., Bulinski (1997) J. Cell Sc. 110:3055-3064; Panda (1997) Proc. Natl. Acad. Sc. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

Particularly preferred agents are compounds with paclitaxel-like activity. These include, but are not limited to paclitaxel and paclitaxel derivatives (paclitaxel-like compounds) and analogues. Paclitaxel and its derivatives are available commercially. In addition, methods of making paclitaxel and paclitaxel derivatives and analogues are well known to those of skill in the art (see, e.g., U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506).

More specifically, the term “paclitaxel” as used herein refers to the drug commercially available as Taxol® (NSC number: 125973). Taxol® inhibits eukaryotic cell replication by enhancing polymerization of tubulin moieties into stabilized microtubule bundles that are unable to reorganize into the proper structures for mitosis. Of the many available chemotherapeutic drugs, paclitaxel has generated interest because of its efficacy in clinical trials against drug-refractory tumors, including ovarian and mammary gland tumors (Hawkins (1992) Oncology, 6: 17-23, Horwitz (1992) Trends Pharmacol. Sci. 13: 134-146, Rowinsky (1990) J. Natl. Canc. Inst. 82: 1247-1259).

Additional microtubule affecting agents can be assessed using one of many such assays known in the art, e.g., a semiautomated assay which measures the tubulin-polymerizing activity of paclitaxel analogs in combination with a cellular assay to measure the potential of these compounds to block cells in mitosis (see Lopes (1997) Cancer Chemother. Pharmacol. 41:37-47).

Generally, activity of a test compound is determined by contacting a cell with that compound and determining whether or not the cell cycle is disrupted, in particular, through the inhibition of a mitotic event. Such inhibition may be mediated by disruption of the mitotic apparatus, e.g., disruption of normal spindle formation. Cells in which mitosis is interrupted may be characterized by altered morphology (e.g., microtubule compaction, increased chromosome number, etc.).

In a preferred embodiment, compounds with possible tubulin polymerization activity are screened in vitro. In a preferred embodiment, the compounds are screened against cultured WR21 cells (derived from line 69-2 wap-ras mice) for inhibition of proliferation and/or for altered cellular morphology, in particular for microtubule compaction. In vivo screening of positive-testing compounds can then be performed using nude mice bearing the WR21 tumor cells. Detailed protocols for this screening method are described by Porter (1995) Lab. Anim. Sci., 45(2):145-150.

Other methods of screening compounds for desired activity are well known to those of skill in the art. Typically such assays involve assays for inhibition of microtubule assembly and/or disassembly. Assays for microtubule assembly are described, for example, by Gaskin et al. (1974) J. Molec. Biol., 89: 737-758. U.S. Pat. No. 5,569,720 also provides in vitro and in vivo assays for compounds with paclitaxel-like activity.

Methods for the safe and effective administration of the above-mentioned microtubule affecting agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, NJ 07645-1742, USA); the disclosure of which is incorporated herein by reference thereto.

The amount and frequency of administration of the compounds of formula (I) and the chemotherapeutic agents and/or radiation therapy will be regulated according to the judgment of the attending clinician (physician) considering such factors as age, condition and size of the patient as well as severity of the disease being treated. A dosage regimen of the compound of formula (I) can be oral administration of from 10 mg to 2000 mg/day, preferably 10 to 1000 mg/day, more preferably 50 to 600 mg/day, in two to four (preferably two) divided doses, to block tumor growth. Intermittent therapy (e.g., one week out of three weeks or three out of four weeks) may also be used.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the patient, and in view of the observed responses of the disease to the administered therapeutic agents.

In the methods of this invention, a compound of formula (I) is administered concurrently or sequentially with a chemotherapeutic agent and/or radiation. Thus, it is not necessary that, for example, the chemotherapeutic agent and the compound of formula (I), or the radiation and the compound of formula (I), should be administered simultaneously or essentially simultaneously. The advantage of a simultaneous or essentially simultaneous administration is well within the determination of the skilled clinician.

Also, in general, the compound of formula (I) and the chemotherapeutic agent do not have to be administered in the same pharmaceutical composition, and may, because of different physical and chemical characteristics, have to be administered by different routes. For example, the compound of formula (I) may be administered orally to generate and maintain good blood levels thereof, while the chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of a compound of formula (I), and chemo-therapeutic agent and/or radiation will depend upon the diagnosis of the attending physicians and their judgement of the condition of the patient and the appropriate treatment protocol.

The compound of formula (I), and chemotherapeutic agent and/or radiation may be administered concurrently (e.g., simultaneously, essentially simultaneously or within the same treatment protocol) or sequentially, depending upon the nature of the proliferative disease, the condition of the patient, and the actual choice of chemotherapeutic agent and/or radiation to be administered in conjunction (i.e., within a single treatment protocol) with the compound of formula (I).

If the compound of formula (I), and the chemotherapeutic agent and/or radiation are not administered simultaneously or essentially simultaneously, then the initial order of administration of the compound of formula (I), and the chemotherapeutic agent and/or radiation, may not be important. Thus, the compound of formula (I) may be administered first followed by the administration of the chemotherapeutic agent and/or radiation; or the chemotherapeutic agent and/or radiation may be administered first followed by the administration of the compound of formula (I). This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the chemotherapeutic agent and/or radiation may be administered first, especially if it is a cytotoxic agent, and then the treatment continued with the administration of the compound of formula (I) followed, where determined advantageous, by the administration of the chemotherapeutic agent and/or radiation, and so on until the treatment protocol is complete.

Thus, in accordance with experience and knowledge, the practicing physician can modify each protocol for the administration of a component (therapeutic agent—i.e., the compound of formula (I), chemotherapeutic agent or radiation) of the treatment according to the individual patient's needs, as the treatment proceeds.

The attending clinician, in judging whether treatment is effective at the dosage At administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radio-logical studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

The following examples illustrate the preparation of some of the compounds of the invention and are not to be construed as limiting the invention disclosed herein. Alternate mechanistic pathways and analogous structures will be apparent to those skilled in the art.

### PREPARATIVE EXAMPLE 1

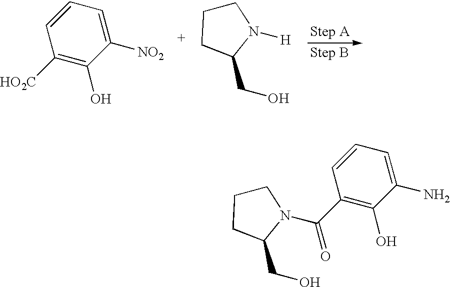

Step A

3-Nitrosalicylic acid (500 mg, 2.7 mmol), 1,3-dicyclohexylcarbodiimide (DCC) (563 mg) and ethyl acetate (10 mL) were combined and stirred for 10 min. (R)-(−)-2-pyrrolidinemethanol (0.27 mL) was added and the resulting suspension was stirred at room temperature overnight. The solid was filtered off and the filtrate was either concentrated down and directly purified or washed with 1 N NaOH. The aqueous phase was acidified and extracted with EtOAc. The resulting organic phase was dried over anhydrous MgSO₄, filtered and concentrated in vacuo. Purification of the residue by preparative plate chromatography (silica gel, 5% MeOH/CH₂Cl₂ saturated with AcOH) gave the desired compound (338 mg, 46%, MH⁺=267).

Step B

The product from Step A above was stirred with 10% Pd/C under a hydrogen gas atmosphere overnight. The reaction mixture was filtered through celite, the filtrate concentrated in vacuo, and the resulting residue purified by column chromatography (silica gel, 4% MeOH/CH₂Cl₂ saturated with NH₄OH) to give the product (129 mg, 43%, MH⁺=237).

### PREPARATIVE EXAMPLE 2

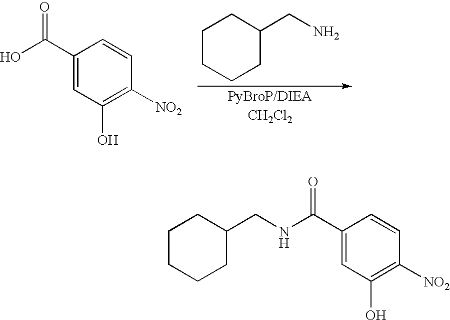

Step A

Cyclohexylmethanamine (0.7 mL, 5.35 mmol, 2.0 eq.) was added in one portion to a stirred solution of 3-hydroxy-4-nitrobenzoic acid (500 mg, 2.68 mmol, 1.0 eq.), diisopropylethylamine (DIEA) (1.4 mL, 8.03 mmol, 3.0 eq.), and bromotripyrrolidinophosphonium hexafluorophosphate (PyBroP), (1.30 g, 2.68 mmol, 1.0 eq.) in anhydrous dichloromethane (25 mL) at room temperature under a nitrogen atmosphere. The mixture was stirred at room temperature for 12 h and diluted with 1.0 M aqueous NaOH solution (50 mL). The mixture was extracted with dichloromethane (4×25 mL) and the organic extracts were discarded. The aqueous phase was acidified with 6.0 M aqueous HCl solution to pH 2 and extracted with ethyl acetate (4×25 mL). The combined organic extracts were washed with brine (50 mL), dried over Na₂SO₄, filtered, and concentrated under house-vacuum at 30° C. The resulting solid (588 mg, 2.11 mmol, 79%, MH⁺=279) was used directly without any further attempts at purification.

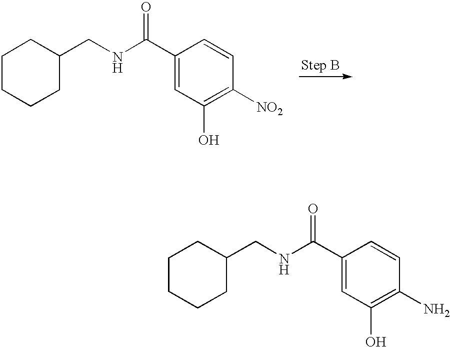

Step B

The aqueous acid solution from Step A above was stirred with 10% Pd/C under a hydrogen gas atmosphere overnight. The reaction mixture was filtered through celite, the filtrate concentrated in vacuo, and the resulting residue purified by column chromatography (silica gel, 4% MeOH/CH₂Cl₂ saturated with NH₄OH) to give the product (319 mg, 62%, MH⁺=249).

Following the procedures set forth in Preparative Examples 1 and 2 but using the carboxylic acid, the amine, and the coupling agent [DCC (Prep. Ex. 1) or PyBrop (Prep. Ex. 2)] listed in Table I below, the indicated amide products were obtained and used without further purification.

TABLE I

| | | | | 1. Coupling Agent |
|---|---|---|---|---|
| | | | | 2. % Yield Step |
| | | | | A, Step B |
| Prep | | | | 3. MH⁺ Step A, |
| Ex. | Carboxylic acid | Amine | Product | Step B |
| 3 | 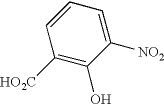 | 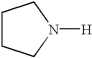 | 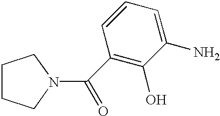 | 1. DCC 2. 50%, 64% 3. 237, 207 |
| 4 | 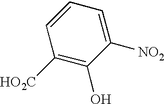 | 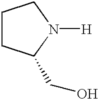 | 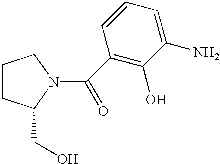 | 1. PyBrop 2. 100%, 31% 3. 267, 237 |
| 5 | 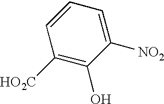 | 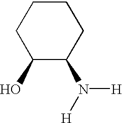 | 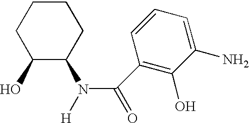 | 1. PyBrop 2. 97%, 27% 3. 281, 251 |
| 6 | 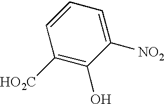 | 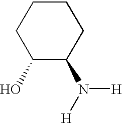 | 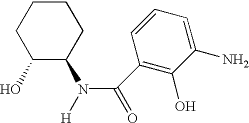 | 1. PyBrop 2. 99%, 14% 3. 281, 251 |
| 7 | 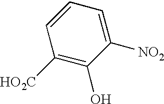 | 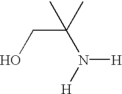 | 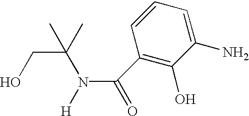 | 1. PyBrop 2. 100%, 26% 3. 255, 255 |
| 8 | 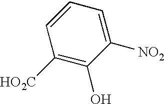 | 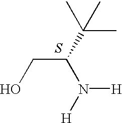 | 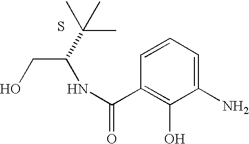 | 1. PyBrop 2. 100, 35% 3. 283, 253 |
| 9 | 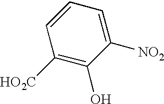 | 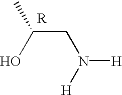 | 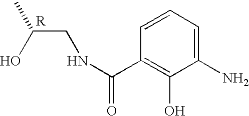 | 1. PyBrop 2. 94%, 15% 3. 241, 211 |
| 10 | 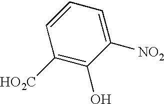 | 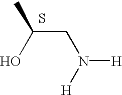 | 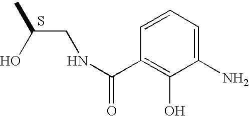 | 1. PyBrop 2. 100%, 33% 3. 241, 211 |
| 11 | 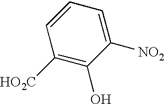 | 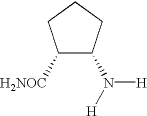 | 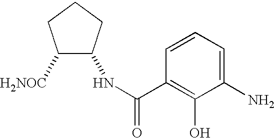 | 1. PyBrop 2. 91%, 29% 3. 294, 264 |
| 12 | 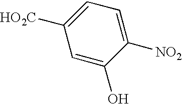 | NH₃ | 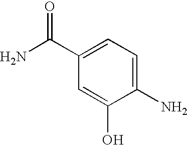 | 1. PyBrop 2. 100%, 38% 3. 183, 153 |
| 13 | 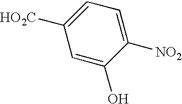 | 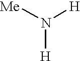 | 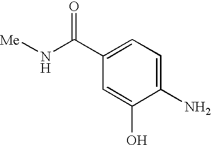 | 1. PyBrop 2. 86%, 64% 3. 197, 167 |
| 14 | 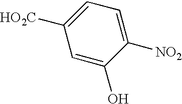 | 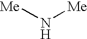 | 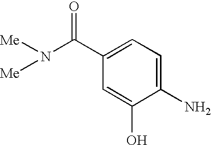 | 1. PyBrop 2. 81%, 68% 3. 211, 181 |
| 15 | 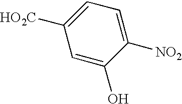 | 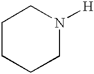 | 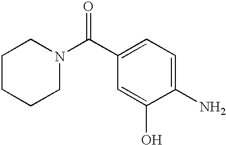 | 1. PyBrop 2. 75%, 39% 3. 251, 221 |
| 16 | 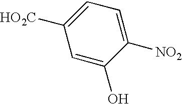 | 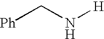 | 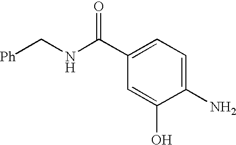 | 1. DCC 2. 33%, 95% 3. 273, 243 |
| 17 | 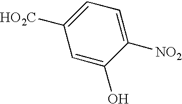 | 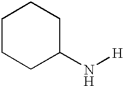 | 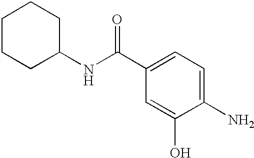 | 1. PyBrop 2. 82%, 47% 3. 265, 235 |
| 18 | 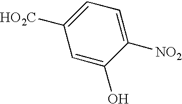 | 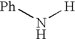 | 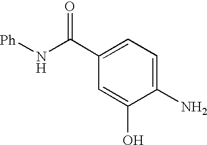 | 1. PyBrop 2. 74%, 37% 3. 259, 229 |
| 19 | 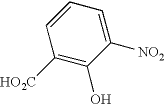 | 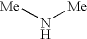 | 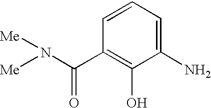 | 1. PyBrop 2. 87%, 86% 3. 211, 181 |

### PREPARATIVE EXAMPLE 20

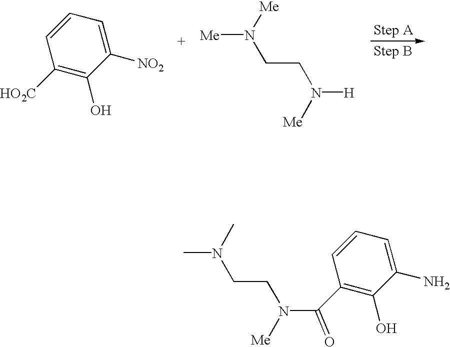

Step A

3-Nitrosalicylic acid (500 mg, 2.7 mmol), DCC (563 mg) and ethyl acetate (10 mL) were combined and stirred for 10 min. N,N-Dimethyl-1,3-propanediamine (0.34 mL) was added and the resulting suspension was stirred at room temperature overnight. The solid was filtered and stirred with 1N HCl. After filtration of the resulting mixture, the aqueous filtrate was used directly in the next reaction.

Step B

The aqueous acid solution from Step A was stirred with 10% Pd/C under a hydrogen gas atmosphere overnight. The reaction mixture was filtered through celite, the filtrate concentrated in vacuo, and the resulting residue purified by column chromatography (silica gel, 4% MeOH/CH₂Cl₂ saturated with NH₄OH) to give the desired product (183 mg, 29%, MH⁺=238).

Following the two-step procedure set forth in Preparative Example 20 but using the carboxylic acid and amine listed in Table II below, the Products were obtained.

TABLE II

| Prep. | | | | 1. % Yield |
|---|---|---|---|---|
| Ex. | Carboxylic acid | Amine | Product | 2. MH⁺ |
| 21 | 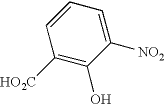 | 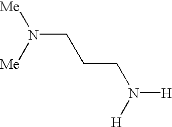 | 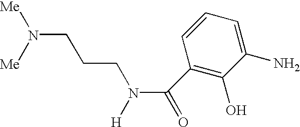 | 1. 39% 2. 238 |
| 22 | 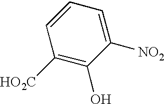 | 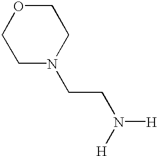 | 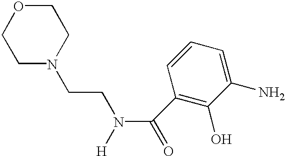 | 1. 19 2. 266 |
| 23 | 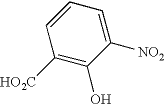 | 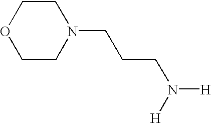 | 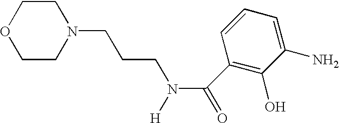 | 1. 29% 2. 280 |
| 24 | 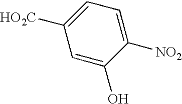 | 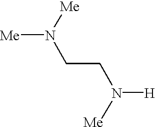 | 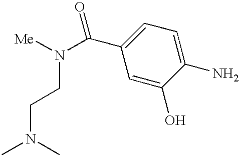 | 1. 52% 2. 238 |

### PREPARATIVE EXAMPLE 25

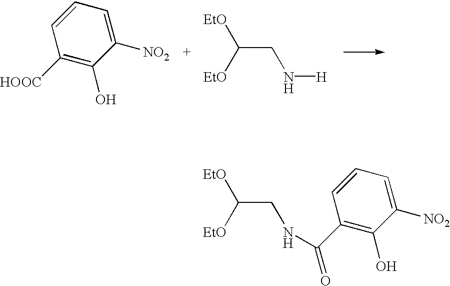

Step A

2,2-diethoxy-ethylamine (4.2 mL) and 3-hydroxy-4-nitrobenzoic acid (5 g) were reacted according to the procedure set forth in Preparative Example 2, Step A (40% yield, MH⁺=299).

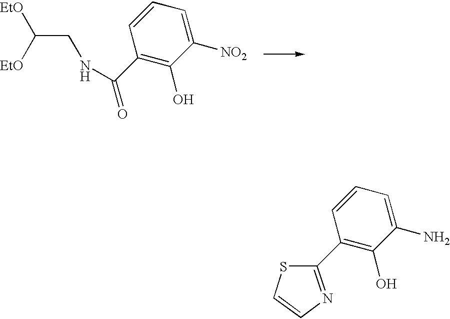

Step B

The product from Step A (806 mg) and P₄S₁₀ (1.5 g) were heated to 130° C., then immediately cooled to room temperature. Water was added and the resulting mixture was filtered. The filtrate was extracted with ethyl acetate and the organic phase was dried over anhydrous MgSO₄, filtered and concentrated in vacuo. Purification of the residue by preparative plate chromatography (silica gel, 2% MeOH/CH₂Cl₂) gave the product (90 mg, 15%).

### PREPARATIVE EXAMPLE 26

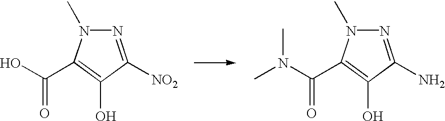

The carboxylic acid as described in the literature (Khimiya Geterotsiklicheskikh Soedinenii 1986, 328-330 [Chemistry of Heterocyclic Compounds 1986, 22, 265-267]) is coupled with dimethylamine and the nitro substituent is reduced according to the procedure outlined in Preparative Example 2, to obtain the pyrazole product shown.

### PREPARATIVE EXAMPLE 27

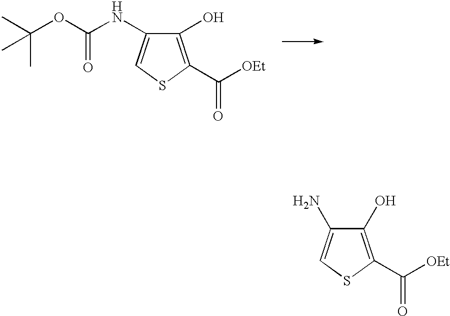

The BOC aminothiophene compound (as prepared in the literature [J. Org. Chem. 1985, 50, 2730-2736]) is treated with HCl in dioxane or trifluoroacetic acid (TFA) in dichloromethane according to procedures known in the art to obtain the thiophene product shown.

### PREPARATIVE EXAMPLE 28

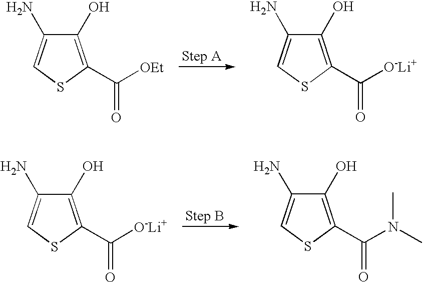

Step A

The title compound from Preparative Example 27 is treated with lithium hydroxide in a suitable solvent according to procedures well established in the art to obtain the lithium carboxylate intermediate shown.

Step B

The lithium carboxylate prepared as described in Step A above is coupled with dimethylamine according to the procedure outlined in Preparative Example 2, to obtain the thiophene product shown.

### PREPARATIVE EXAMPLE 29

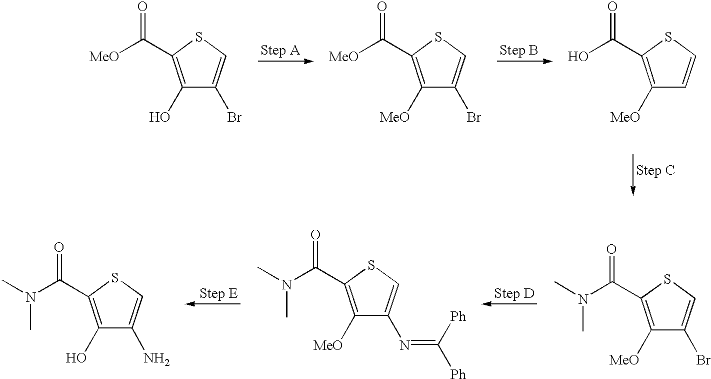

### Step A

Methyl-3-hydroxy-4-bromo-2-thiophenecarboxylate (10.0 g, 42.2 mmol) was dissolved in 250 mL of acetone. Potassium carbonate (30.0 g, 217.4 mmol) was added followed by a solution of iodomethane (14.5 mL, 233.0 mmol). The mixture was heated to reflux and continued for 6 h. After cooled to room temperature, the mixture was filtered, the solid material was rinsed with acetone (-200 mL). The filtrate and rinsing were concentrated under reduced pressure to a solid, further dried on high vacuum, yielding 13.7 g (100%) of methyl-3-methoxy4-bromo-2-thiophenecarboxylate. (MH⁺=251.0).

### Step B

Methyl-3-methoxy-4-bromo-2-thiophenecarboxylate (13.7 g), available from step A, was dissolved in 75 mL of THF, and added with a 1.0 M sodium hydroxide aqueous solution (65 mL, 65.0 mmol). The mixture was stirred at room temperature for 24 h. A 1.0 M hydrogen chloride aqueous solution was added dropwise to the mixture until pH was approximately 2. The acidic mixture was extracted with CH₂Cl₂ (100 mL×2, 50 mL). The combined organic extracts were washed with brine (40 mL), dried with Na₂SO₄, and concentrated under reduced pressure to a solid, 10.0 g (100%, over two steps) of 3-methoxy-4-bromo-2-thiophenecarboxylic acid (MH⁺⁼237.0).

### Step C

To a stirred solution of 3-methoxy4-bromo-2-thiophenecarboxylic acid (6.5 g, 27.4 mmol) in 140 mL of CH₂Cl₂, obtained from step B, was added bromotripyrrolidinophosphonium hexafluorophosphate (PyBrop, 12.8 g, 27.5 mmol), a 2.0 M solution of dimethyl amine in THF (34.5 mL, 69.0 mmol), and diisopropylethyl amine (12.0 mL, 68.7 mmol). After 3 d, the mixture was diluted with 100 mL of CH₂Cl₂, and washed with a 1.0 M sodium hydroxide aqueous solution (30 mL×3) and brine (30 mL). The organic solution was dried with Na₂SO₄, filtered, and concentrated to an oil. This crude oil product was purified by flash column chromatography, eluting with CH₂Cl₂-hexanes (1:1, v/v). Removal of solvents afforded a solid, further dried on high vacuum, yielding 6.76 g (93%) of N,N′-dimethyl-3-methoxy4-bromo-2-thiophenecarboxamide (MH⁺=265.0, M+2=266.1).

### Step D

An oven dried three-neck round bottom flask was equipped with a refluxing condenser, charged sequentially with palladium acetate (95 mg, 0.42 mmol), (R)-2,2′-Bis(diphenylphosphino)-1,1′-binaphthyl (BINAP) (353 mg, 0.57 mmol), cesium carbonate (9.2 g, 28.33 mmol), and N,N′-dimethyl-3-methoxy-4-bromo-2-thiophenecarboxamide (3.74 g, 14.2 mmol, from step C). The solid mixture was flushed with nitrogen (“degass via house vacuum/refill with nitrogen”, three cycles). Toluene (95 mL) was added to the solid mixture followed by benzophenone imine (3.6 mL, 21.5 mmol). The mixture was heated to reflux and continued for 10 h. A second batch of palladium acetate (95 mg, 0.42 mmol) and (R)-BINAP (353 mg, 0.57 mmol) in 5 mL of toluene was added. Refluxing was continued for 14 h. The third batch of palladium acetate (30 mg, 0.13 mmol) and (R)-BINAP (88 mg, 0.14 mmol) was added, and reaction continued at 110° C. for 24 h. The mixture was cooled to room temperature, diluted with ether (50 mL), filtered through a layer of Celite, rinsing with ether. The filtrate and rinsing were concentrated under reduced pressure to an oil, which was purified twice by flash column chromatography using CH₂Cl₂ and CH₂Cl₂-MeOH (200:1) as eluents. Removal of solvents afforded 4.1 g (79%) of the amido-thiophene diphenylimine product as a solid (MH⁺=365.1).

### Step E

To a stirred solution of thiophene imine (5.09 g, 13.97 mmol), obtained from step D, in 140 mL of CH₂Cl₂ at −78° C. was added dropwise a 1.0 M solution of boron tribromide in CH₂Cl₂. The mixture was stirred for 3 h while the temperature of the cooling bath was increased slowly from −78° C. to −15° C. 100 mL of H₂O was added, the mixture was stirred at room temperature for 30 min, then the two layers were separated. The organic layer (as A) was extracted with H₂O (30 mL×2). The aqueous layer and aqueous extracts were combined, washed with CH₂Cl₂ (30 mL), and adjusted to pH-8 using a saturated NaHCO₃ aqueous solution. The neutralized aqueous solution was extracted with CH₂Cl₂ (100 mL×3), the extracts were washed with brine, dried with Na₂SO₄, and concentrated under reduced pressure to a solid, 1.49 g of N,N′-dimethyl-3-hydroxy-4-amino-2-thiophenecarboxamide (first crop). The previous separated organic layer A and organic washing were combined, stirred with 30 mL of a 1.0 M HCl aqueous solution for 1 h. The two layers were separated, the aqueous layer was washed with CH₂Cl₂ (30 mL) and adjusted to pH˜8 using a saturated NaHCO₃ aqueous solution, and the separated organic layer and organic washing were combined as organic layer B. The neutralized aqueous solution was extracted with CH₂Cl₂ (30 mL×4), the extracts were washed with brine, dried by Na₂SO₄, and concentrated under reduced pressure to give 0.48 g of a solid as the second crop of the titled product. Organic layer B from above was washed with brine, and concentrated to an oil, which was separated by preparative TLC (CH₂Cl₂-MeOH =50:1) to afford 0.45 g of a solid as the third crop of the titled product. The overall yield of the product, N,N′-dimethyl-3-hydroxy-4-amino-2-thiophenecarboxamide, is 2.32 g (89%) (MH⁺=187.0).

### PREPARATIVE EXAMPLE 30

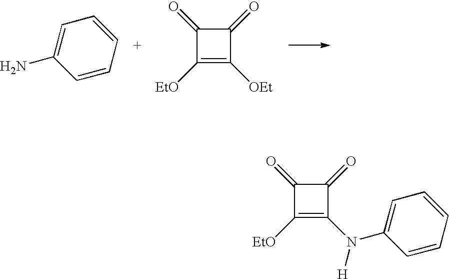

Aniline (12 mL) dissolved in absolute EtOH (150 mL) was added dropwise over 6 hours to a stirred ethanolic (150 mL) solution of diethylsquarate (20 g) at 0° C. After stirring at room temperature overnight, the reaction mixture was filtered and the filtrate concentrated in vacuo. The resulting residue was washed with cold EtOH and ether to give the above product (23.5 g, 92%, MH⁺=218).

### PREPARATIVE EXAMPLE 31

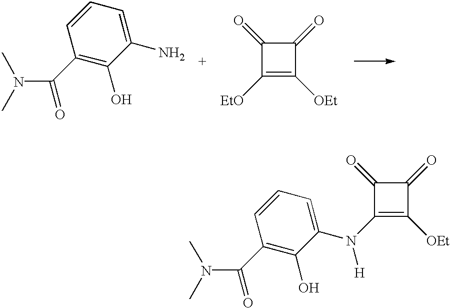

The compound from Preparative Example 19 (14.6 g) dissolved in absolute EtOH (100 mL) was added dropwise over 4 hours to a stirred ethanolic (100 mL) solution of diethylsquarate (19 mL, 128 mmol). After 5 days, the reaction mixture was concentrated in vacuo, and the resulting residue purified by column chromatography (silica gel, 0-5% MeOH/CH₂Cl₂) to give the product (65%, MH⁺=305, mp=178.6° C.).

### PREPARATIVE EXAMPLE 32

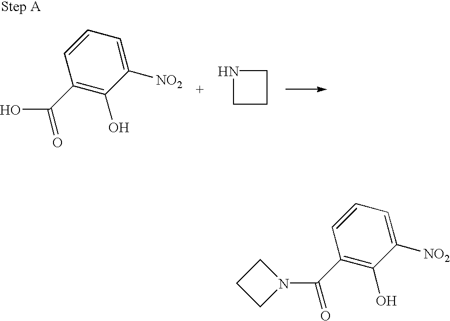

3-Nitrosalicylic acid (1.0 g, 5.5 mmol) was dissolved in ethyl acetate (20 mL). 1,3-Dicyclohexylcarbodiimide (0.568 g, 2.8 mmol) was added and the mixture was stirred for approximately 10 minutes and cooled to 0° C. During this time a precipitate formed. Azetidine (0.39 mL, 5.8 mmol) was added and the reaction was stirred overnight and allowed to warm to room temperature. After this time the reaction was cooled to 0° C. and filtered. The collected solid was washed with chilled ethyl acetate. The filtrate was concentrated and purified by column chromatography (80% EtOAc/Hex) to give the product (476 mg, 39.0%).

¹H NMR (300 MHz, CDCl₃) δ 2.40 (m, 2H), 4.38 (m, 4H), 6.97 (m, 1H), 7.62 (d, 1H), 8.12 (d, 1H), 12.88 (m, 1H) ppm.

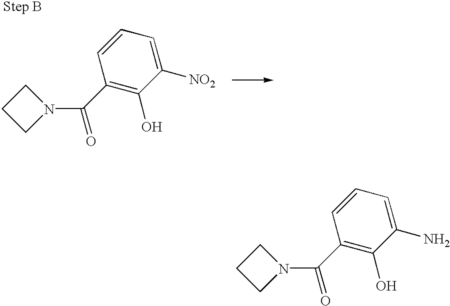

The nitro compound (0.48 g, 2.1 mmol) from Preparative Example 32 Step A was dissolved in methanol (25 ml) and stirred with 10% Pd/C under a hydrogen gas atmosphere overnight. The reaction mixture was filtered through celite, the filtrate concentrated in vacuo to give the product (344 mg, 90%).

¹H NMR (300 MHz, CDCl₃) δ 2.52 (m, 2H), 4.57 (bs, 4H), 6.75 (m, 1H), 6.90 (m, 2H), 12.71 (bs, 1H) ppm.

### PREPARATIVE EXAMPLE 33

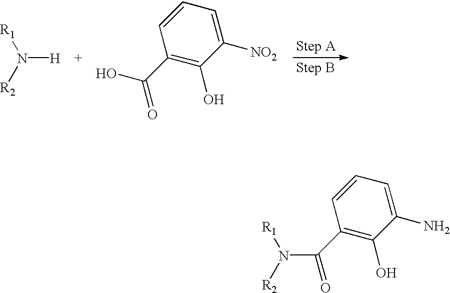

Following the two-step procedure set forth in Preparative Example 32 but using the carboxylic acid and amine listed in the Table III below, the Products were obtained.

TABLE III

| Prep. | | | | 1. % |
|---|---|---|---|---|
| Ex. | Carboxylic acid | Amine | Product | Yield |
| 33 | 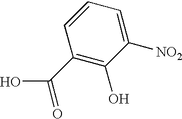 | 2M dimethylamine in THF | 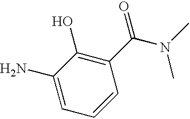 | 1. 75% |
| 34 | 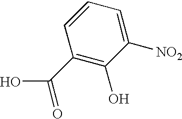 | 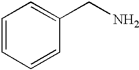 | 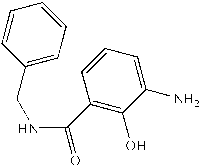 | 1. 70% |
| 35 | 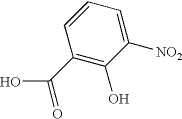 | 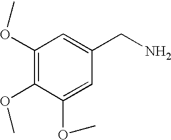 | 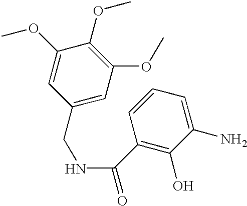 | 1. 68% |
| 36 | 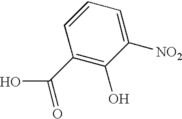 | 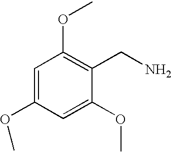 | 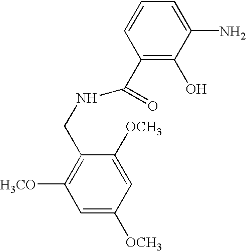 | 1. 39% |
| 37 | 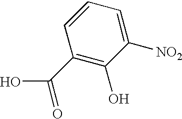 | 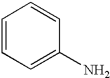 | 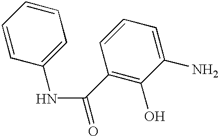 | 1. 66% |
| 38 | 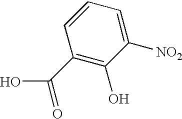 | 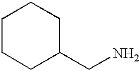 | 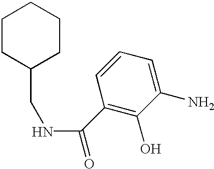 | 1. 60% |
| 39 | 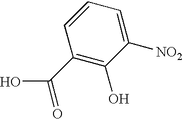 | 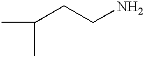 | 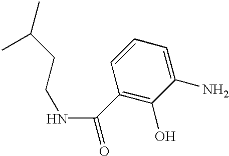 | 1. 51% |
| 40 | 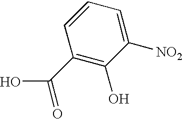 | 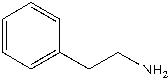 | 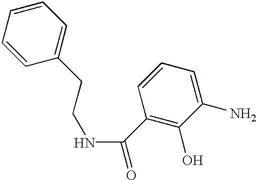 | 1. 97% |
| 41 | 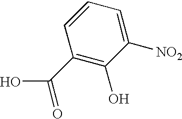 | 2M methylamine in THF | 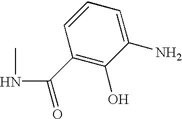 | 1.90% |
| 42 | 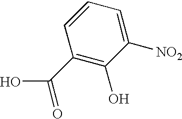 | 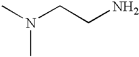 | 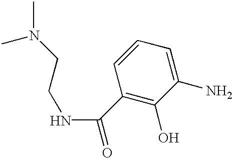 | 1. 81% |
| 43 | 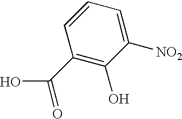 | 2M ethylamine in THF | 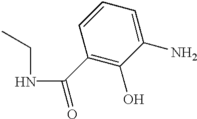 | 1. 64% |
| 44 | 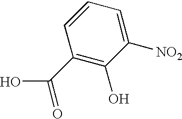 | 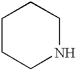 | 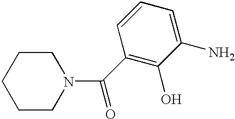 | 1. 26% |
| 45 | 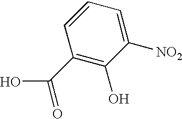 | 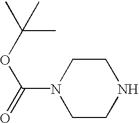 | 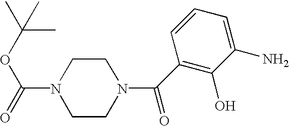 | 1. 19% |
| 46 | 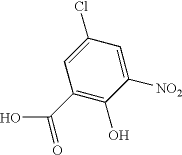 | 2M dimetylamine in THF | 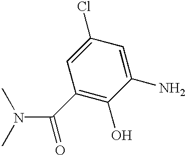 | 1. 85% |
| 47 | 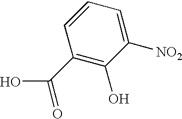 | 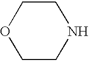 | 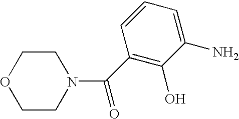 | 1. 39% |

### PREPARATIVE EXAMPLE 48

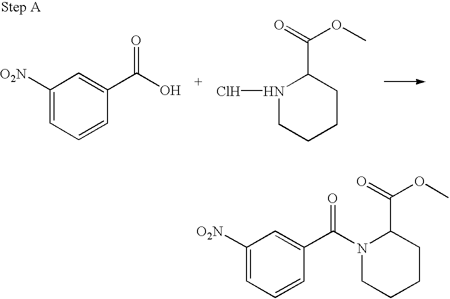

3-Nitrobenzoic acid (1.004 g, 6.0 mmol) was combined with N,N-diisopropylethylamine (6.25 mL, 36.0 mmol) in dichloromethane (60 mL). Bromo-tris-pyrrolodino-phosphonium hexafluorophosphate (PyBrOP), (2.80 g, 6.0 mmol) was added to the solution and the mixture was stirred for ten minutes. Methyl picolinate hydrochloride (1.08 g, 6.0 mmol) was added to the mixture and the reaction was stirred overnight. After this time the reaction was concentrated and product was isolated by column chromatography (1:9 EtOAc/DCM). Product was isolated as a yellow solid and used without further purification (1.66 g, 95%).

¹H NMR (300 MHz, CDCl₃) δ 1.46 (m, 2H), 1.65 (m, 1H), 1.90 (m, 2H), 2.39 (m, 1), 3.32 (m, 1H), 3.53 (m, 1H), 3.81 (s, 3H), 5.50 (m, 1H), 7.62 (m, 1H), 7.78 (m, 1H), 8.31 (m, 2H)ppm.

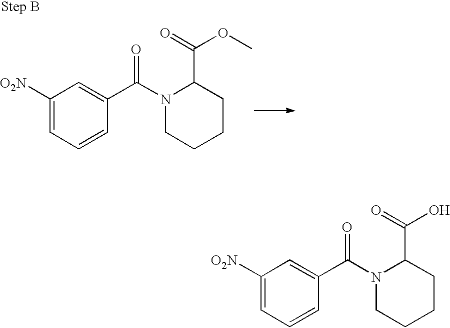

The methyl ester (1.79 g, 6.1 mmol) was dissolved in dioxane/water (20 mL/15 mL) at room temperature. Lithium hydroxide (0.258 g, 6.2 mmol) was added to the solution. After a few hours more lithium hydroxide was added (0.128 g, 3.0 mmol) and the reaction was stirred for another hour. After this time the reaction was concentrated and then taken up in water. The solution was extracted two times with ether. The aqueous phase was then acidified and extracted three times with ethyl acetate. The organic fractions were then dried over sodium sulfate, filtered and ;10 concentrated. Product was isolated by column chromatography (95% EtOAc/Hex, 0.05% HOAc) to give the product (1.66 g, 98%)

¹H NMR (300 MHz, CDCl₃) δ 1.49 (m, 2H), 1.68 (m, 1H), 1.82 (m, 2H), 2.44 (m, 1H) 3.32 (m, 1H), 3.58 (m, 1H), 5.57 (m, 1H), 7.65 (m, 1H), 7.80 (m, 1H), 8.32 (m, 2H), 10.04 (bs, 1 Hppm).

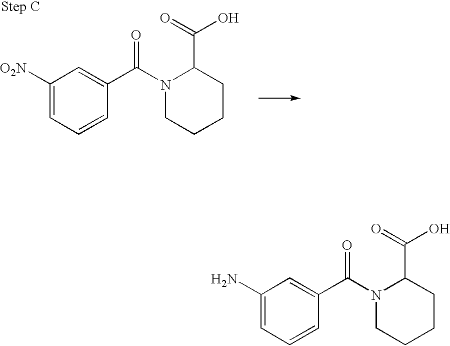

The nitro compound was dissolved in an excess of methanol (20 mL) and covered by a blanket of argon. 5% Palladium on carbon was added (catalytic) and a hydrogen balloon was attached to the flask. The atmosphere of the system was purged under vacuum and replaced with hydrogen. This step was repeated for a total of three times. The reaction was then stirred under hydrogen overnight. After this time the balloon was removed and the solution was filtered through celite followed by several rinses with methanol. The filtrate was concentrated and dried on the vacuum line to provide the desired aniline product (1.33 g, 90%).

¹H NMR (300 MHz, CDCl₃) δ 1.40 (m, 2H), 1.50 (m, 1H), 1.68 (m, 2H), 2.33 (m, 1H) 3.18 (m, 1H), 3.62 (m, 1H), 5.39 (m, 1H), 6.12 (bs, 2H), 6.75 (m, 2H), 7.12 (m, 1H)ppm.

Mass Spectra, calculated: 248, found: 249.1 (M+1)⁺

### PREPARATIVE EXAMPLES 49-51

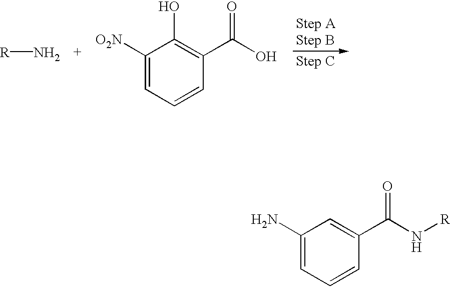

Following the three-step procedure set forth in Preparative Example 48 but using the carboxylic acid and amine listed in Table IV below, the following products were obtained.

TABLE IV

| Prep. | | | | |
|---|---|---|---|---|
| Ex. | Carboxylic acid | Amine | Product | % Yield |
| 49 | 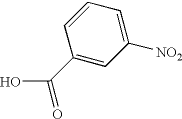 | 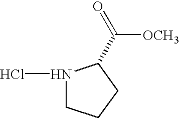 | 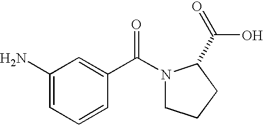 | 43% |
| 50 | 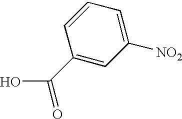 | 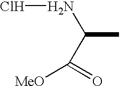 | 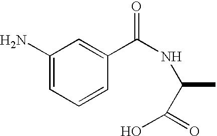 | 36% |
| 51 | 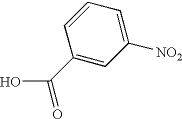 | 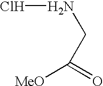 | 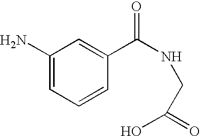 | 7.6% |

### PREPARATIVE EXAMPLE 52

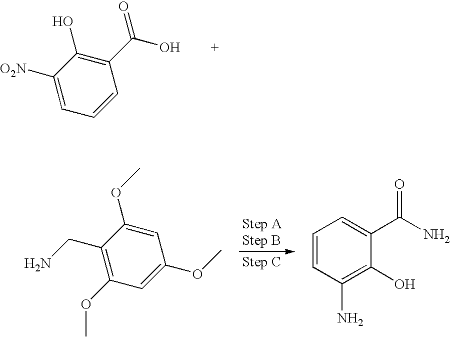

Step A

3-Nitrosalicylic acid (2.009, 10.9 mmol) was combined with 1,3-diisopropylcarbodiimide (1.71 mL, 10.9 mmol) and 4-(dimethylamino)pyridine (catalytic) in dichloromethane (150 mL) and stirred for a few minutes. 2,4,6-Trimethoxybenzylamine hydrochloride (0.664 g, 2.8 mmol) was added along with N,N-diisopropylethylamine (1.88 mL, 10.8 mmol). The reaction was stirred overnight. After this time the reaction was concentrated and purified by column chromatography (111 Hexane/EtoAc) to give the product (1.62 g, 41%).

¹H NMR (300 MHz, CDCl₃) δ 3.83 (m, 9H), 4.72 (d, 2H), 6.17 (s, 2H), 7.01 (m, 1H), 7.88 (m, 1H), 8.18 (dd, 1H), 8.25 (dd, 1H)ppm.

Mass Spectra, calculated: 362.11, found: 362.9 (M+1)⁺

Step B

3-Nitrosalicylic-2,4,6-trimethoxybenzylamide (0.146 g, 0.4 mmol) from Step A above was combined with a solution of trifluoroacetic acid/dichloromethane (1:1, :2; 5 mL). The reaction was stirred for 45 minutes. After this time, TLC (30% E/H) indicated that no starting material was present. The reaction was concentrated and dried on the vacuum line. The material was purified by column chromatography (5% MeOH/CH₂Cl₂) to give the product (0.06 g, 80%).

¹H NMR (300 MHz, CDCl₃) δ 7.16 (m, 1H), 8.28 (m, 1H), 8.49 (m, 1H), 12.26 (s, 1H)ppm.

Step C

The nitro compound (0.32 g, 1.6 mmol) from Step B above was dissolved in an excess of methanol (40 mL) and covered by a blanket of argon. 5% Palladium on carbon was added (catalytic) and a hydrogen balloon was attached to the flask. The atmosphere of the system was purged under vacuum and replaced with hydrogen. This step was repeated for a total of three times. The reaction was then stirred under hydrogen overnight. After this time the balloon was removed and the solution was filtered through Celite followed by several rinses with methanol. The filtrate was concentrated and dried on the vacuum line to provide the desired aniline product (0.17 g, 70%). ¹H NMR (300 MHz, d4-MeOH) 66.63 (m, 1H), 6.88 (m, 1H), 7.07 (d, 1H)ppm.

### PREPARATIVE EXAMPLE 53

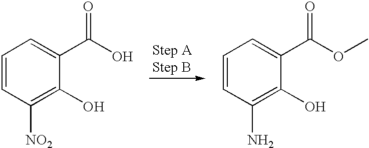

Step A

3-Nitrosalacylic acid (2.00 g, 10.9 mmol) was combined with 1,3-diisopropylcarbodiimide (1.71 mL, 10.9 mmol) and 4-(dimethylamino)pyridine (catalytic) in dichloromethane (150 mL). Methanol was added and the reaction was stirred for 2 hrs. After this time the reaction was concentrated and purified by column chromatography (3/1H/E) to give the methyl ester (0.32 g, 15%).

¹H NMR (300 MHz, d₆-DMSO)δ 3.92 (s, 3H), 7.11 (dd, 1H), 8.05 (d, 1H), 8.19 (d, 1H), 11.46 (s, 1H)ppm.

Step B

The nitro compound (0.32 g, 1.6 mmol) was dissolved in an excess of methanol (40 mL) and covered by a blanket of argon. 5% Palladium on carbon was added (catalytic) and a hydrogen balloon was attached to the flask. The atmosphere of the system was purged under vacuum and replaced with hydrogen. This step was repeated three times. The reaction was stirred under hydrogen overnight. After this time, the balloon was removed and the solution was filtered through Celite followed by several rinses with methanol. The filtrate was concentrated and dried on the vacuum line to provide the desired aniline product (0.18 g, 68%).

¹H NMR (300 MHz, d₆-DMSO)δ 3.92 (bs, 3H), 6.70 (dd, 1H), 6.89 (dd, 1H), 7.22 (d, 1H), 10.85 (bs, 1H)ppm.

Mass Spec.: calculated 167, found 168.0 (M+1)⁺

### PREPARATIVE EXAMPLE 54

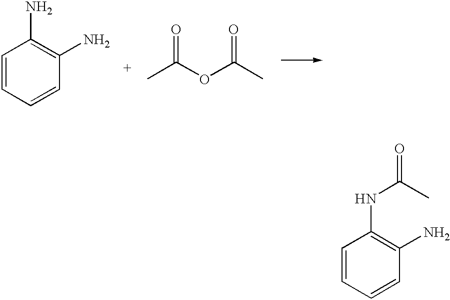

Phenylenediamine (2.20 g, 20 mmol) was dissolved in pyridine (20 mL) and chilled to 0° C. Acetic anhydride (1.89 mL, 20 mmol) and dichloromethane (10 mL) were mixed and added dropwise to the solution over 15 min. The reaction was stirred for 1 hr at 0° C. then warmed to ambient. After 2 hr, the solvent was evaporated. The residue was azeotroped with toluene and dried under vacuum to give the above compound as a solid (2.8 g, 93%).

¹H NMR (300 MHz, d₆-DMSO)δ 2.15 (s, 3H), 4.80-5.05 (bs, 2H), 6.62 (m, 1H), 6.80 (d, 1H), 7.00 (t, 1H), 7.23 (d, 1H), 9.20 (s, 1H)ppm.

### PREPARATIVE EXAMPLE 55

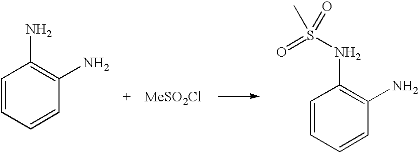

Phenylenediamine (5.0 g, 46 mmol) was dissolved in dichloromethane (50 mL). A solution of methanesulfonyl chloride (3.6 mL, 46 mmol) in dichoromethane (50 mL) was added slowly with stirring. After 16 hr, precipitate was filtered and discarded. The remaining solution was evaporated to give the above compound as a solid (5.5 g, 65%).

Mass Spectra, calculated: 186.0, found 186.9 (M+1)⁺

### PREPARATIVE EXAMPLE 56

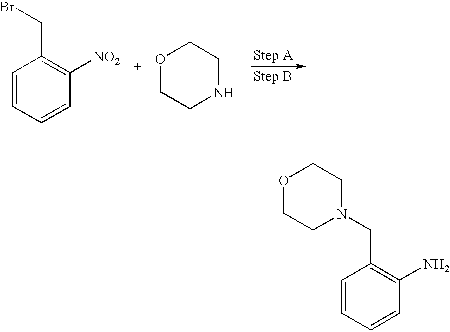

Step A

2-Nitrobenzyl bromide (5.0 g, 0.0231 mol), THF (50 mL) and morpholine (6.05 g, 0.0694 mol) were added to a sealed tube. The reaction mixture was heated to reflux overnight. Removal of the solvent, was followed by addition of water (400 mL)and extraction with DCM (3×80 mL). The combined organic phase were dried over Na₂SO₄, concentrated and purified by column chromatography (25% EtOAc/HEX) to give the above compound (5.07 g, 99%).

¹H NMR (300 MHz, d-CHCl₃) δ 2.5 (m, 4H), 3.8 (m, 4H), 3.9 (s, 2H), 7.5 (t, 1H), 7.7 (m, 2H), 7.9 (d, 1H)ppm.

Step B

The nitro compound (4.57 g, 0.0206 mol) from step A was dissolved in methanol (100 mL) and stirred with 10% Pd/C under a hydrogen gas atmosphere overnight. The reaction mixture was filtered through celite, the filtrate was concentrated and purified by column chromatography (EtOAc/HEX/Et₃N 20/60/1) to give the above compound (3.14 g, 79%).

¹H NMR (300 MHz, d-DMSO)δ 2.5 (m, 4H), 3.5 (s, 2H), 3.7 (m, 4H), 5.4 (s, 2H), 6.6 (t, 1H), 6.7 (d, 1H), 7.1 (m, 2H)ppm.

### PREPARATIVE EXAMPLE 57

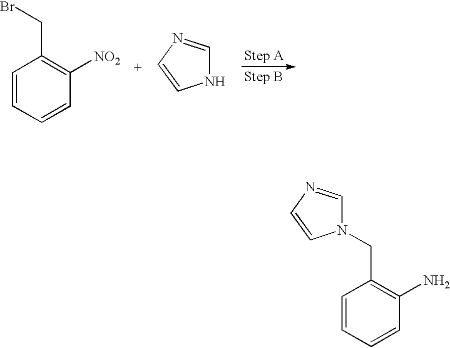

Step A

2-Nitrobenzyl bromide (5.0 g, 0.0231 mol), THF (50 mL) and imidazole (4.72 g, 0.0694 mol) were added to a sealed tube. The reaction mixture was heated to reflux overnight. The solvent was evaporated to give a residue which was taken up in water (400 mL) and extracted with EtOAc (3×80 mL). The combined organic phases were dried over Na₂SO₄, concentrated in vacuo to give the desired compound (4.07 g, 87%).

¹H NMR (300 MHz, d-DMSO) 65.7 (s, 2H), 6.9 (d, 1H), 7.1 (d, 1H), 7.3 (s, 1H), 7.7 (t, 1H), 7.8 (m, 2H), 8.2 (d, 1H)ppm.

Step B

The nitro compound (2.23 g, 0.0110 mol) from step A was dissolved in methanol (50 mL) and stirred with 10% Pd/C under a hydrogen gas atmosphere overnight. The reaction mixture was filtered through celite, the filtrate was concentrated and purified by column chromatography (DCM/MeOH/Et₃N 20/2/1) to give the above compound (1.77 g, 93%).

¹H NMR (300 MHz, d-DMSO)δ 5.2 (s, 2H), 5.3 (s, 2H), 6.6 (t, 1H), 6.8 (d, 1H), 6.9 (d, 1H), 7.0 (s, 1H), 7.1 (t, 1H), 7.2 (s, 1H), 7.8 (s, 1H)ppm.

### PREPARATIVE EXAMPLE 58

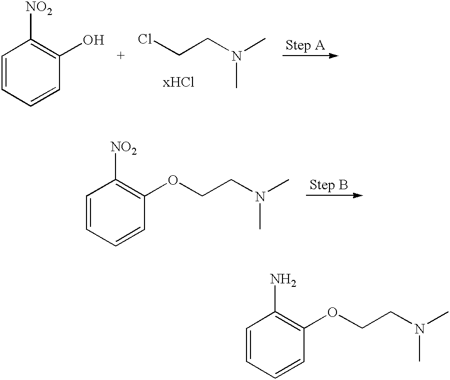

Step A

2-Nitrophenol (4.32 g, 30 mmol) was dissolved in EtOH (40 mL) and then added to a solution of 2-(dimethylamino)ethyl chloride hydrochloride (5.56 g, 34 mmol) and KOH (3.5 g, 63.0 mmol) in BuOH (50 mL) and DMF (10 mL). The reaction mixture was heated to reflux overnight. After cooling to room temperature, the majority of the solvent was evaporated under reduced pressure. The remaining residue was put into water (400 mL) and extracted with EtOAc (3×100 mL). Subsequently, the combined organic phases were washed with 5% NaOH (3×100 mL) and dried over sodium sulfate. The solution was concentrated and purified by column chromatography (10% MeOH/DCM) to give the product (1.35 g, 21%).

H NMR (300 MHz, CDCl₃) δ 2.48 (s, 6H), 2.93(2, 2H), 4.36 (t, 2H), 7.16 (dd, 1H), 7.20 (d, 1H), 7.63 (dd, 1H), 7.97 (d, 1H)ppm.

Step B

The nitro compound (1.35 g, 6.43 mmol) from step A was dissolved in MeOH (50 mL) and shaken with 10% Pd/C under a hydrogen gas atmosphere at 10 psi for 3 h. The reaction mixture was filtered through celite, the filtrate concentrated in vacuo to give the above compound (980 mg, 85%) after column chromatography (DCM/MeOH/NH₄OH=20/1/0.1).

H NMR (300 MHz, CDCl₃) δ 2.46 (s, 6H), 2.95 (t, 2H), 3.60 (bs, 2H), 4.21 (t, 2H), 6.81 (m, 2H), 6.95 (m, 2H)ppm.

### PREPARATIVE EXAMPLE 59

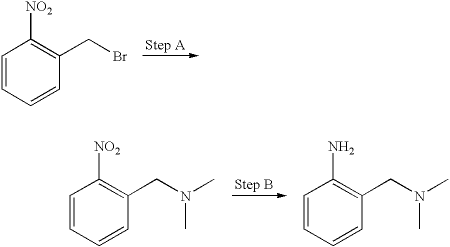

Step A

2-Nitrobenzyl bromide (2.0 g, 9.3 mmol) was dissolved in DCM (50 mL). After addition of dimethylamine (2.0N in THF, 9.3 mL, 18.6 mmol), the reaction mixture was stirred overnight. Subsequently, the mixture was put into water (200 mL) and extracted with DCM (3×100 mL). The combined organic phases were dried over sodium sulfate. The solution was concentrated in vacuo to give the pure compound (540 mg, 32%) after column chromatography (DCM/MeOH/NH₄OH=20/1/0.1).

H NMR (300 MHz, CDCl₃) δ 2.36 (s, 6H), 3.73 (s, 2H), 7.21 (t, 1H), 7.37 (d, 1H), 7.43 (t, 1H), 7.52 (d, 1H)ppm.

Step B

The nitro compound (500 mg, 2.78 mmol) from step B was dissolved in MeOH (50 mL) and stirred with 10% Pd/C under a hydrogen gas atmosphere overnight. The reaction mixture was filtered through celite, the filtrate concentrated in vacuo to give the above compound (400 mg, −80%) after column chromatography (DCM/MeOH/NH₄OH=20/1/0.1).

H NMR (300 MHz, CDCl₃) δ 2.32 (s, 6H), 3.62 (s, 2H), 4.11 (bs, 2H), 6.42 (m, 2H), 6.85 (m, 2H)ppm.

### PREPARATIVE EXAMPLE 60

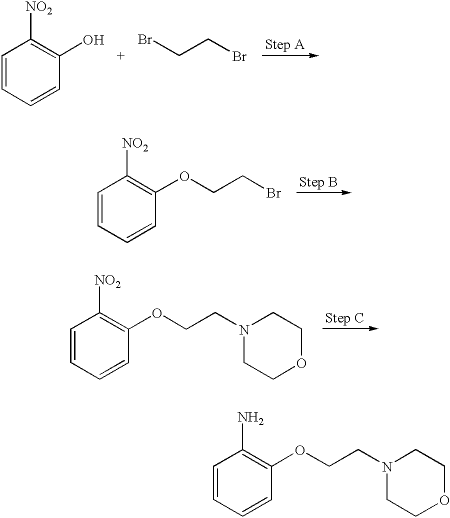

Step A

2-Nitrophenol (5.0 g, 36.0 mmol) was put into water (20 mL). After addition of NaOH (1.44 g, 36.0 mmol) and dibromoethylene (27.0 g, 144.0 mmol) the reaction mixture was refluxed for 40 h. After cooling to room temperature, the mixture was put into water (400 mL) and extracted with EtOAc (3×100 mL). Subsequently, the combined org. phases were washed with 5% NaOH (3×100 mL) and dried over sodium sulfate. The solution was concentrated and purified by column chromatography (75% EtOAc/Pentane) to give the product (3.4 g, 38%).

H NMR (300 MHz, CDCl₃) δ 3.79 (t, 2H), 4.57 (t, 2H), 7.20 (m, 2H), 7.65 (dd, 1H), 7.97 (d, 1H)ppm.

Step B

The nitrobromide (1.7 g, 6.9 mmol) was dissolved in THF (20 mL). After addition of morpholine (1.81 mL, 20.7 mmol), the reaction mixture was refluxed over night. After cooling to room temperature, the reaction mixture was put into water (300 mL) and extracted with DCM (3×100 mL). The combined org. phases were dried over sodium sulfate. The solution was concentrated and purified by column chromatography (CH₂Cl₂/MeOH/NH₄OH=20/1/0.1) to give the product (1.73 g, 99%).

H NMR(300 MHz, CDCl₃) δ 2.74 (t, 4H), 3.00 (t, 2H), 3.84 (t, 4H), 4.39 (t, 2H), 7.18 (dd, 1H), 7.20 (d, 1H), 7.63 (dd, 1H), 7.93 (d, 1H)ppm.

Step C

The nitro compound (1.71 g, 6.78 mmol) from step B was dissolved in MeOH (50 mL) and stirred with 10% Pd/C under a hydrogen gas atmosphere overnight. The reaction mixture was filtered through celite, the filtrate concentrated in vacuo to give the desired compound (1.43 g, 95%) after column chromatography (DCM/MeOH/NH4OH=20/1/0.1).

H NMR (300 MHz, CDCl₃) δ 2.71 (t, 4H), 2.92 (t, 2H), 3.84 (t, 4H), 4.00 (bs, 2H), 4.28 (t, 2H), 6.82 (m, 2H), 6.94 (m, 2H)ppm.

### PREPARATIVE EXAMPLE 61

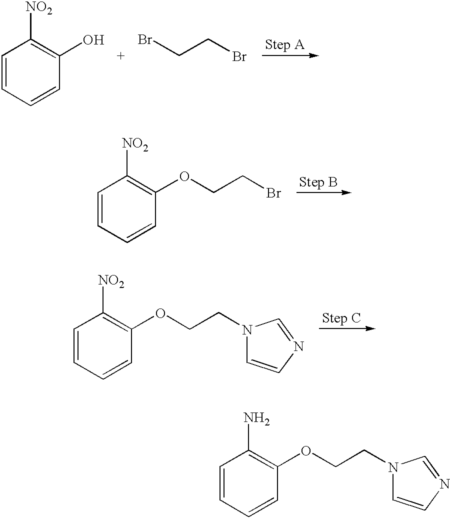

Step A

This reaction follows step A of Preparative Example 60.

H NMR (300 MHz, CDCl₃) δ 3.79 (t, 2H), 4.57 (t, 2H), 7.20 (m, 2H), 7.65 (dd, 1H), 7.97 (d, 1H)ppm.

Step B

The nitrobromide from Step A(1.7 g, 6.9 mmol) was dissolved in THF (20 mL). After addition of imidazole (1.41 g, 20.7 mmol) the reaction mixture was refluxed over night. After cooling to room temperature, the reaction mixture was put into water (300 mL) and extracted with CH₂Cl₂ (3×100 mL). The combined org. phases were dried over sodium sulfate. The solution was concentrated and purified by column chromatography (CH₂Cl₂/MeOH/NH4OH=10/1/0.1) to give the product (1.25 g, 78%).

H NMR (300 MHz, CDCl₃) δ 4.41 (t, 2H), 4.56 (t, 2H), 7.06 (d, 1H), 7.18(s+dd, 2H), 7.26 (s, 1H), 7.63 (dd, 1H), 7.74 (s, 1H), 7.99 (d, 1H)ppm.

Step C

The nitro compound (1.23 g, 5.28 mmol) from step B of Preparative Example 61 was dissolved in MeOH (50 mL) and stirred with 10% Pd/C under a hydrogen gas atmosphere for 3 h. The reaction mixture was filtered through celite, the filtrate concentrated in vacuo to give the above compound (1.01 g, 94%) after column chromatography (DCM/MeOH/NH4OH=10/1/0.1).

H NMR (300 MHz, CDCl₃) δ 3.41 (bs, 2H), 4.38 (t, 2H), 4.48 (t, 2H), 6.82 (m, 3H), 6.95 (m, 1H), 7.17 (s, 1H), 7.21 (s, 1H), 7.62 (d, 1H)ppm.

### PREPARATIVE EXAMPLE 62

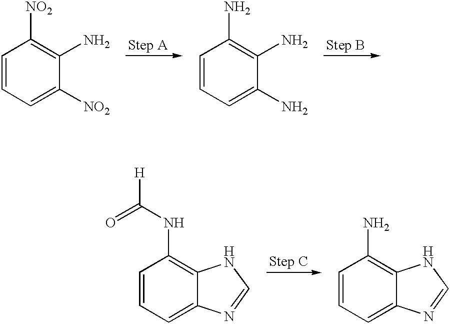

Step A

2,6-Dinitroaniline (10.0 g, 55.0 mmol) and tin(II)chloride dihydrate (111.0 g, 492.0 mmol) were solved in conc. HCl (170 mL). The reaction mixture was refluxed for 5 h and then allowed to cool to room temperature. After sitting over night, the precipitate was filtered off and subsequently dissolved in 10% NaOH (50 mL). The solvent was evaporated under reduced pressure and the remaining residue was extracted with EtOAc (10×80 mL). The solvent of the combined extracts was removed and the resulting residue (2.5 g crude) was used in step B without any further purification.

Step B

The crude material from step A was dissolved in 96% formic acid (10 mL). After refluxing for 1 h, the solution was evaporated to dryness. After addition of water (10 mL), the pH of the acidic solution was adjusted to 7 using concentrated ammonium hydroxide solution. The resulting precipitate was collected, dried, and used in the next step without further purification.

Step C

The crude formic amide from step B was dissolved in 10% HCl (25 mL) and refluxed for 30 min. Removal of the solvent was followed by addition of 10% NaOH (6 mL). After evaporation of the solvent, the resulting residue was extracted with EtOH (4×50 mL). The solution was concentrated and purified by column chromatography (DCM/MeOH/NH4OH=5/1/0.1) to give the final product (1.23 g, 18% over 3 steps).

H NMR (300 MHz, d₆-DMSO)δ 5.38 (bs, 2H), 6.44 (d, 1H), 7.82 (d, 1H), 6.99 (t, 1H), 8.11 (s, 1H), 12.30 (bs, 1H)ppm.

### PREPARATIVE EXAMPLE 63

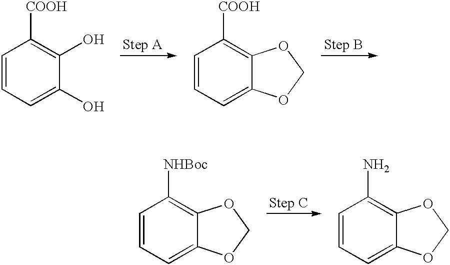

Step A

2,3-Dihydroxybenzoic acid (15.0 g, 97.3 mmol) was suspended in water (30 mL). After addition of a solution of KOH (16.4 g, 292 mmol) in water (70 mL) diiodomethane (8.1 mL, 100.2 mmol) was added. The reaction mixture was heated to 100° C. for 5 days or until almost all of the diiodo compounds disappeared. The remaining rest of the dihalogen starting material was co-evaporated with some water. The solution was acidified with concentrated HCl to yield a precipitate. The crude acetal was collected and recrystallized once from EtOH to yield crystals (7.0 g, 43%).

H NMR (300 MHz, d₆-DMSO) 66.21 (s, 2H), 6.99 (dd, 1H), 7.22 (d, 1H), 7.39 (d, 1H), 13.07 (bs, 1H)ppm.

Step B

The recrystallized material (2.0 g, 12.0 mmol) from step A was refluxed for 10 min in a mixture of dioxane (35 mL) and tert-butylalcohol (10 min). After the mixture was allowed to cool to room temperature, diphenylphosphoryl azide (2.6 mL, 12.0 mmol) and DIEA (1.8mL, 13.0 mmol) were added in one batch. The reaction mixture was refluxed for 8 h and the dioxane was removed under reduced pressure. The reaction mixture was put into water (200 mL) and extracted with CH₂Cl₂ (3×100 mL). The combined organic phases were dried over sodium sulfate. The solution was concentrated and finally purified by column chromatography to give the product (2.28 g, 80%).

H NMR (300 MHz, CDCl₃) δ 1.44 (s, 9H), 6.21 (s, 2H), 6.56 (m, 2H), 6.81 (t, 1H), 7.23 (s, 1H)ppm.

Step C

The carbamate (2.28 g, 9.6 mmol) from step B was suspended in EtOH (50 mL). To the suspension was added 5N HCl (50 mL). Stirring over night resulted in a clear solution. The solvent was removed under reduced pressure and the residue was dissolved in water (200 mL). The solution was neutralized with KOH and then extracted with EtOAc (3×100 mL). The combined organic phases were dried over sodium sulfate, concentrated and finally purified by column chromatography (DCM/MeOH/NH4OH=20/1/0.2) to yield the desired product (1.05 g, 80%).

H NMR (300 MHz, CDCl₃) δ 3.48 (bs, 2H), 6.03 (s, 2H), 6.43 (d, 1H), 6.46 (d, 1H), 6.79 (t, 1H)ppm.

### PREPARATIVE EXAMPLE 64

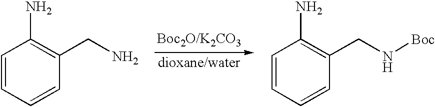

2-Aminobenzyl amine (5.0 g, 41.0 mmol) was dissolved in a mixture of dioxane/water (30 mL each). After addition of Boc-anhydride (8.94 g, 41.0 mmol) and potassium carbonate (8.5 g, 61.5 mmol), the mixture was stirred over night. The solution was put into water (300 mL) and extracted with EtOAc (3×100 mL). The combined org. phases were dried over sodium sulfate, concentrated and finally purified by column chromatography (25% EtOAc/Pentane) to yield the desired product (7.28 g, 80%).

Mass Spec.: calculated 222.1, found 223.0 (M+1)⁺

### PREPARATIVE EXAMPLE 65

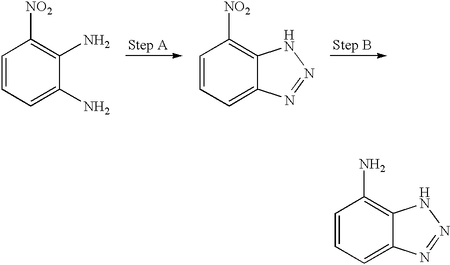

Step A

2,3-Diaminonitrophenol (4.0 g, 26.1 mmol) was dissolved in AcOH (200 mL). After addition of sodium nitrite (2.25 g, 32.7 mmol), the reaction mixture was heated to 60° C. for 3 h. The solvent was removed under reduced pressure and the residue was put into water (200 mL) and extracted with EtOAc (3×00 mL). The combined org. phases were dried over sodium sulfate, concentrated, and finally purified by column chromatography (50% EtOAc/Pentane) to yield the desired product (3.42 g, 80%).

H NMR (300 MHz, d₆-DMSO) δ 7.78 (dd, 1H) 8.60 (d, 1H), 8.73 (d, 1H)ppm.

Step B

The nitro triazole (3.4 g, 20.9 mmol) from step A was dissolved in MeOH (50 mL) and stirred with 10% Pd/C under a hydrogen gas atmosphere over night. The reaction mixture was filtered through celite and washed very thoroughly with MeOH. Finally, the filtrate was concentrated in vacuo to give the desired compound (2.38 g, 85%)

H NMR (300 MHz, d₆-DMSO) 65.99 (bs, 2H), 6.51 (d, 1H), 6.93 (d, 1H), 7.22 (dd, 1H)ppm.

### PREPARATIVE EXAMPLE 66

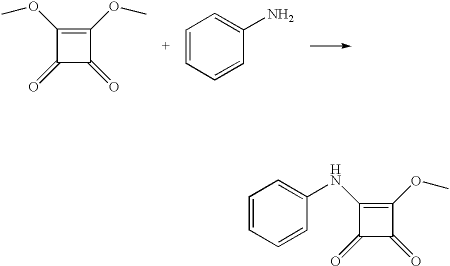

3,4-Dimethoxy-3-cyclobutene-1,2-dione (1.30 g, 9.2 mmol) was dissolved in methanol. Aniline (0.84 mL, 9.2 mmol) was added dropwise to the solution. The reaction was stirred at room temperature for 16 hours. After this time a solid formed which was determined to be the desired product. The solid was collected by filtration and dried under vacuum (1.8 g, 96%).

¹H NMR (300 MHz, d₆-DMSO)δ 4.39 (s, 3H), 7.12 (m, 1H), 7.35 (m, 4H), 10.75 (bs, 1H)ppm.

### PREPARATIVE EXAMPLES 67-83

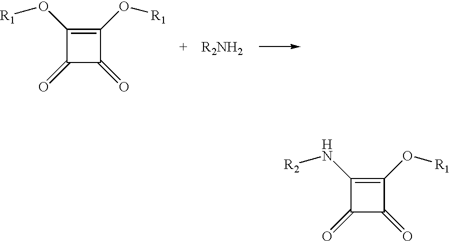

Following the procedure set forth in Preparative Example 66, but using the alkoxysquarate and the amine or aniline (R₂-NH₂) listed in Table V below, the following products were obtained.

TABLE V

| Prep. | | R₂—NH₂ or Aniline | | 1. % Yield |
|---|---|---|---|---|
| Ex. | R₁ | from Prep Ex | Product | 2. (M + 1)⁺ |
| 67 | Et | 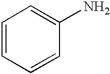 | 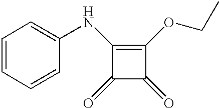 | 1. 95% 2. 218.0 |
| 68 | Et | 54 | 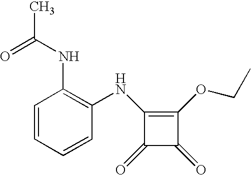 | 1. 95% 2. 274.9 |
| 69 | Et | 55 | 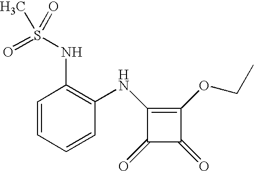 | 1. 50% 2. 311.0 |
| 70 | Me | 65 | 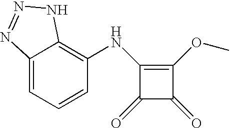 | 1. 77% 2. 245.1 |
| 71 | Me | 63 | 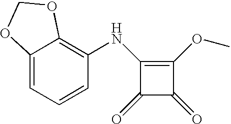 | 1. 82% 2. 248.1 |
| 72 | Me | 59 | 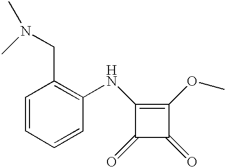 | 1. 71% 2. 261.0 |
| 73 | Me | 62 | 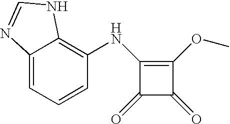 | 1. 73% 2. 244.1 |
| 74 | Me | 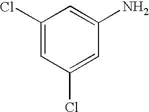 | 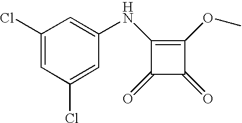 | 1. 62% 2. 272.1 |
| 75 | Me | 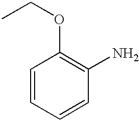 | 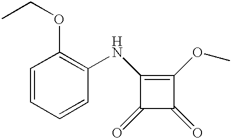 | 1. 78% 2. 248.1 |
| 76 | Me | 64 | 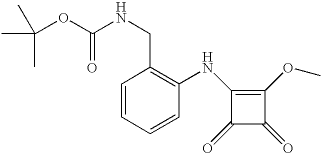 | 1. 78% 2. 332.1 |
| 77 | Me | 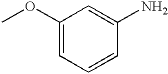 | 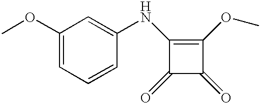 | 1. 87% 2. 234.1 |
| 78 | Me | 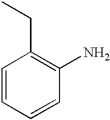 | 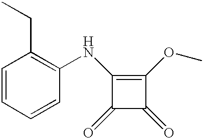 | 1. 85% 2. 232.2 |
| 79 | Me | 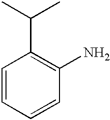 | 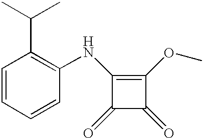 | 1. 85% 2. 246.1 |
| 80 | Me | 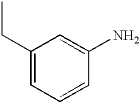 | 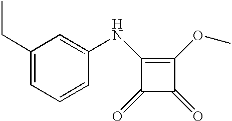 | 1. 80% 2. 232.2 |
| 81 | Me | 56 | 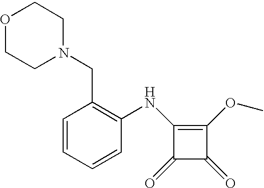 | 1. 82% 2. 303.1 |
| 82 | Me | 58 | 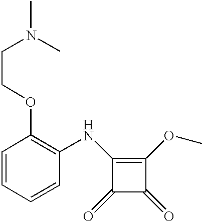 | 1. 68% 2. 291.2 |
| 83 | Me | 57 | 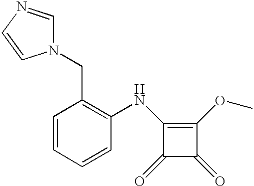 | 1. 73% 2. 284.0 |

### PREPARATIVE EXAMPLE 84

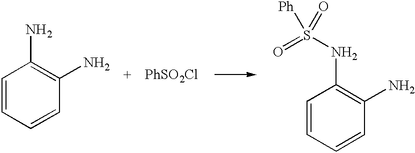

1,2-Phenylenediamine (5.0 g, 0.0462 mol) was dissolved in methylene chloride ). Benzenesulfonyl chloride (5.6 mL, 0.0439 mol) was added dropwise and the n was stirred for 72 hours. After this time, TLC (5% MeOH/DCM) indicated the n was complete. The reaction was filtered to remove any solid material and the was washed with methylene chloride. The filtrate was concentrated and purified by column chromatography (3% MeOH/DCM). The desired product (2.28 g, 0.0092 mol, 20%) was isolated as a solid.

¹H NMR (300 MHz, CD₃OD) δ 6.40 (m, 2H), 6.73 (d, 1H), 6.94 (m, 1H), 7.46 (m, 2H), 7.58 (m, 1H), 7.68 (m, 2H)ppm.

MS-APCI: calculated 248.06, found 248.9 (M+1)⁺

### PREPARATIVE EXAMPLE 85

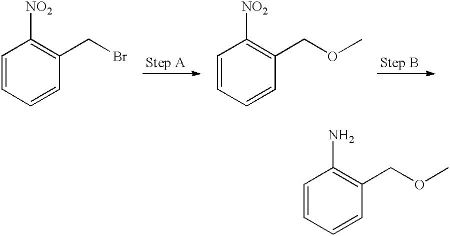

Step A:

2-Nitrobenzyl bromide (5.18 g, 0.024 mol) was dissolved in EtOH (25 mL). NaOMe (11.0 mL 25% wt in MeOH, 0.048 mol) was added drop wise under argon atmosphere. After stirred at room temperature for 1 h, sat. sodium hydrogen carbonate solution (200 mL) was added. The mixture was extracted with chloroform (3×80 mL). The combined organic phases were washed with sat. sodium hydrogen carbonate solution (80 mL), water (80 mL), brine (80 mL) and dried over sodium sulfate. Concentration and purification by column chromatography (20% EtOAc/HEX) gave the desired compound (3.70 g, 92%).

¹H NMR (300 MHz, d-CHCl₃) δ 3.60 (s, 3H), 4.95 (s, 2H), 7.55 (t, 1H), 7.78 (t, 1H), 7.90 (d, 1H), 8.20 (d, 1H)ppm.

Step B:

An ethanolic suspension of Raney-Ni was added to a stirred solution of the nitro compound (3.00 g, 0.018 mol) from Step A in EtOAc/EtOH (10 mL/10 mL) under argon atmosphere. The mixture was refluxed overnight and then filtered through celite. The filtrate was concentrated and purified by column chromatography (25% EtOAc/HEX) to give the desired compound (1.65 g, 67%).

¹H NMR (300 MHz, d-CHCl₃) δ 3.45 (s, 3H), 4.38 (bs, 2H), 4.60 (s, 2H), 6.82 (t, 2H), 7.22 (m, 2H)ppm.

MS(MH⁺): 137.08, found 137.9.

### PREPARATIVE EXAMPLE 86

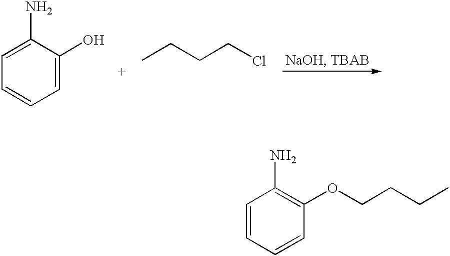

2-Aminophenol (1.269, 0.012 mol), sodium hydroxide (1.84 g, 0.046 mol), and tetrabutylammonium bromide (0.19 g, 0.58 mmol) were mixed at room temperature and stirred for 10 minutes. 1-Chlorobutane (1.2 mL, 0.012 mol) was added and the mixture was heated to 60° C. for 8 hours. The mixture was purified directly by column chromatography (25% EtOAc/HEX) to give the desired compound (0.95 g, 50%).

¹H NMR (300 MHz, d-CHCl₃) δ 1.08 (t, 3H), 1.62 (m, 2H), 1.90 (m, 2H), 4.05 (t, 2H), 4.23 (bs, 2H), 6.85 (m, 4H)ppm. MS(MH⁺): 165.12, found 166.1.

### PREPARATIVE EXAMPLE 87

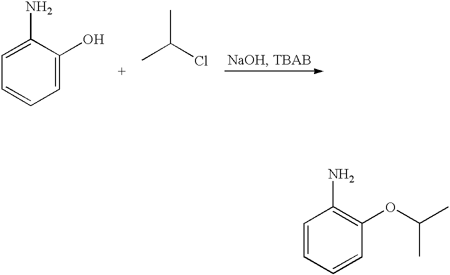

2-Aminophenol (5.0 g, 0.046 mol), sodium hydroxide (7.33 g, 0.183 mol) and tetrabutylammonium bromide (0.74 g, 2.29 mmol) were mixed at room temperature and stirred for 10 minutes. 2-Chloropropane (4.2 mL, 0.046 mol) was added and the mixture was heated to 60° C. for 8 hours. The mixture was purified directly by column chromatography (25% EtOAc/HEX) to give the desired compound (0.92 g, 13%).

¹H NMR (300 MHz, d-CHCl₃) δ 1.45 (d, 6H), 4.03 (bs, 2H), 4.60 (m, 1H), 6.93 (m, 4H)ppm.

MS(MH⁺): 151.10, found 152.1.

### PREPARATIVE EXAMPLE 89

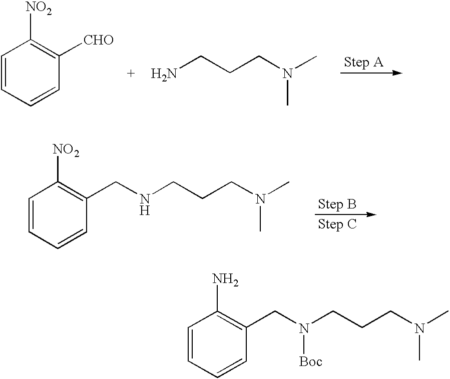

Step A:

2-Nitrobenzaldehyde (2.0 g, 0.0132 mol), 1,2-dichloroethane (100 mL) and 3-(dimethylamino)propylamine (1.83 mL, 0.0145 mol) were stirred for 1 h. After addition of sodium triacetoxyborohydride (4.20 g, 0.0198 mol), the reaction mixture was stirred overnight. Addition of 1 N NaOH (100 mL) was followed by extraction of EtOAc (3×100 mL) and drying over sodium sulfate The solution was concentrated and purified by column chromatography (DCM/MeOH/Et₃N 40/4/1) to give the desired compound (1.62 g, 52%).

¹H NMR (300 MHz, d-DMSO) 61.58 (m, 2H), 2.20 (s, 6H), 2.28 (t, 2H), 2.58 (m, 2H), 3.15 (s, 1H), 4.00 (s, 2H), 7.58 (t, 1H), 7.78 (m, 2H), 8.00 (d, 1H)ppm.

MS(MH⁺): 237.15, found 238.2.

Step B:

The nitro compound (1.62 g, 0.0068 mol) from Step A was dissolved in THF (50 mL) and water (50 mL). Di-tert-butyl dicarbonate (1.49 g, 0.0068 mol) and sodium carbonate (1.44 g, 0.0136 mol) were added and the reaction mixture was stirred overnight. Addition of water (100 mL) was followed by extraction with EtOAc (3×50 mL). The combined organic phases were dried over sodium sulfate, concentrated and purified by column chromatography (DCM/MeOH/NH₄OH 40/4/1) to give the desired compound(1.38 g, 60%).

¹H NMR (300 MHz, d-DMSO) 61.40 (d, 9H), 1.68 (m, 2H), 2.18 (s, 6H), 2.23 (t, 2H), 3.32 (d, 2H), 4.78 (s, 2H), 7.42 (d, 1H), 7.26 (t, 1H), 7.83 (t, 1H), 8.15 (d, 1H).

MS: 337.20, found 338.1.

Step C:

The nitro compound from Step B was dissolved in MeOH (25 mL) and stirred with a catalytic amount of 5% Pd/C under hydrogen atmosphere overnight. The reaction mixture was filtered through celite, the filtrate concentrated and purified by column chromatography (4% Et₃N/EtOAc) to give the desired compound (1.16 g, 92%).

¹H NMR (300 MHz, d-DMSO)δ 1.53 (s, 9H), 1.62 (m, 2H), 2.08 (s, 6H), 2.20 (t, 2H), 3.15 (t, 2H), 4.33 (s, 2H), 5.20 (s, 2H), 6.58 (t, 1H), 6.72 (d, 1H), 7.03 (m, 2H)ppm.

MS(MH⁺): 307.23, found 308.1.

### PREPARATIVE EXAMPLE 90

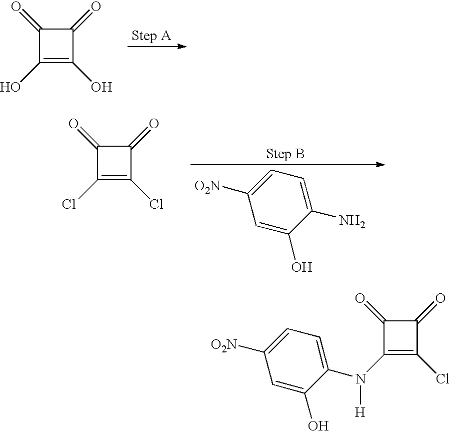

Step A

Squaric acid (1.14 g, 10 mmol) suspended in thionyl chloride (8 mL) and N,N-dimethylformamide (0.050 mL) was refluxed under argon for 2 hr. The solvent was evaporated, and the residue was dissolved in diethyl ether and washed with ice water. The ether phase was dried with sodium sulfate and evaporated to give an oil. The oil was stored under vacuum for one hour.

Step B

The dichloride from Step A was dissolved in 1,2-dichlorobenzene (5 mL) and mixed with 2-amino-5-nitrophenol (1.54 g, 10 mmol). A precipitate formed after 10 min. The solution was stirred for 2 more hours. The solid was collected by filtration and washed with 1,2-dichlorobenzene.

¹H NMR (300 MHz, CD₃OD) δ 7.29 (d, 1H), 7.87 (m, 2H)ppm.

MS-: calculated 268.0, found 267.0 (M−1)⁻

### PREPARATIVE EXAMPLE 91

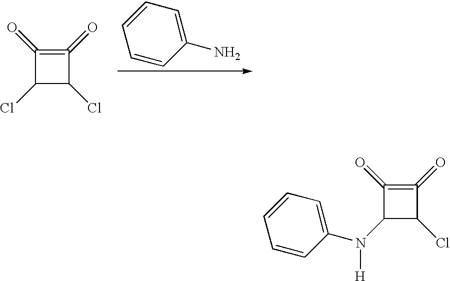

The dichloride (1.13 g, 7.5 mmol) from Preparative Example 90, Step A was dissolved in tetrahydrofuran (5 mL) and chilled to 0 C. Aniline (0.697 mL, 7.5 mmol) was dissolved in tetrahydrofuran (5 mL), chilled to 0 C, and added dropwise to the dichloride solution over 10 min. The mixture was warmed to ambient while stirring for one hour. The solvent was evaporated to give a solid. The solid was taken up in acetonitrile, filtered, and washed with more acetonitrile. A powder was recovered (0.91 g, 59% yield).

Mass Spec.: calculated 207.0, found 209.2 (M+2)⁺

### EXAMPLE 1

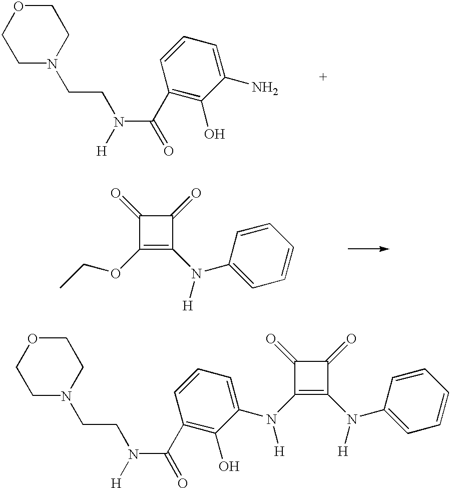

The product from Preparative Example 22 (93 mg), the ethoxysquarate compound from Preparative Example 30 (75 mg), triethylamine (0.12 mL) and absolute ethanol (5 mL) were heated at reflux overnight. The reaction mixture was concentrated in vacuo and the residue was purified by preparative plate chromatography (silica gel, 8% MeOH/CH₂Cl₂ saturated with NH₄OH) to give the product (51 mg, 34%, MH⁺=437).

### EXAMPLES 2-27

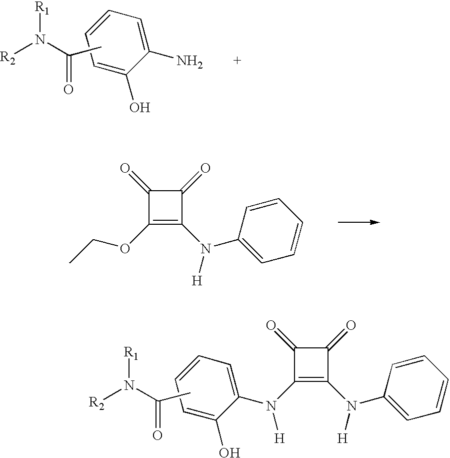

Following the procedure described for Example 1, the Products listed in Table VI below were prepared using the amine from the Preparative Example indicated (or the commercially available aniline illustrated) and the ethoxy squarate from Preparative Example 30.

TABLE VI

| | | | 1. Yield (%) |
|---|---|---|---|
| | Amine from | | 2. MH⁺ |
| Example | Prep Ex | Product | 3. mp (° C.) |
| 2 | 3 | 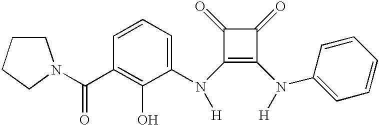 | 1. 39% 2. 378 3. 172.3 |
| 3 | 1 | 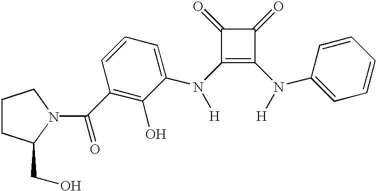 | 1. 30% 2. 408 3. 180.8 |
| 4 | 4 | 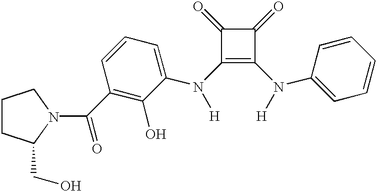 | 1. 23% 2. 408 3. 160.4 |
| 5 | 5 | 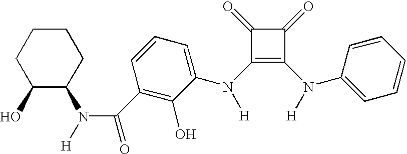 | 1. 42% 2. 422 3. 172.3 |
| 6 | 6 | 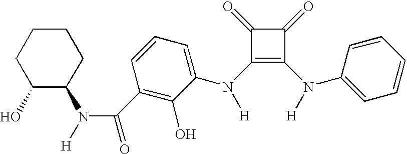 | 1. 51% 2. 422 3. 203.1 |
| 7 | 7 | 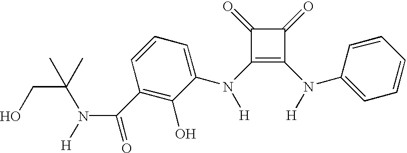 | 1. 72% 2. 396 3. 180.6 |
| 8 | 8 | 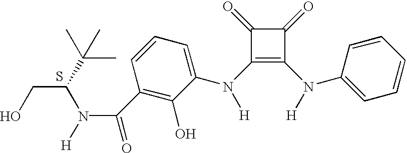 | 1. 80% 2. 424 3. 180.2 |
| 9 | 9 | 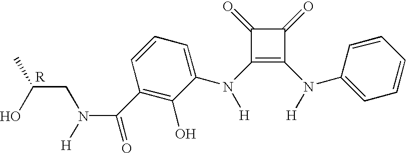 | 1. 78% 2. 382 3. 154.6 |
| 10 | 10 | 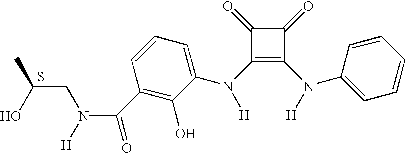 | 1. 1.21% 2. 382 3. 218.6 |
| 11 | 11 | 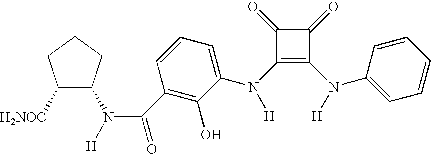 | 1. 74% 2. 435 3. 186.3 |
| 12 | 20 | 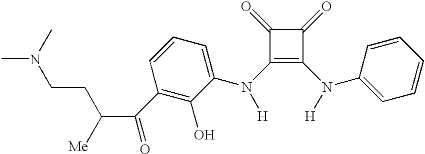 | 1. 74% 2. 409 3. 163.6 |
| 13 | 21 | 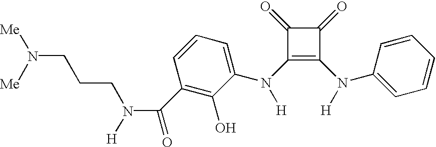 | 1. 57% 2. 409 3. 176.8 |
| 14 | 23 | 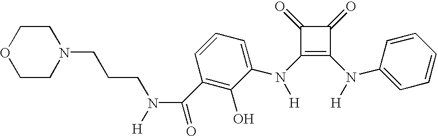 | 1. 75% 2. 451 3. 164.4 |
| 15 | 25 | 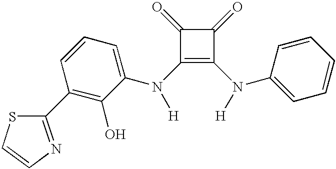 | 1. 17% 2. 364 3. 292.7 |
| 16 | 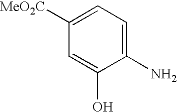 | 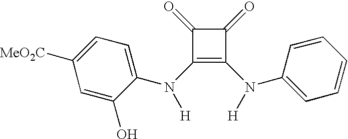 | 1. 43% 2. 339 |
| 17 | 24 | 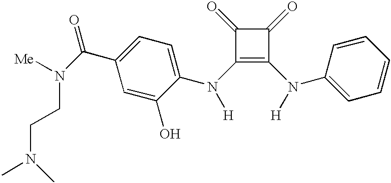 | 1. 14% 2. 409 3. 175.2 |
| 18 | 12 | 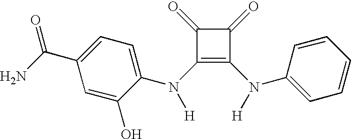 | 1. 81% 2. 324 3. 290-300 |
| 19 | 13 | 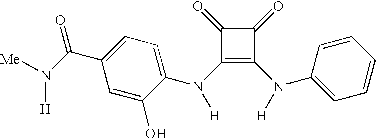 | 1. 83% 2. 338 3. >300 |
| 20 | 14 | 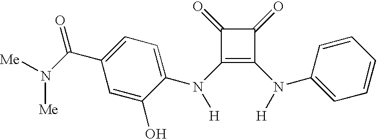 | 1. 82% 2. 352 3. >300 |
| 21 | 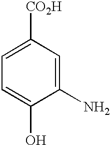 | 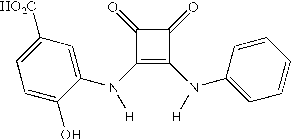 | 1. 56% 2. 325 3. 298.7 |
| 22 | 15 | 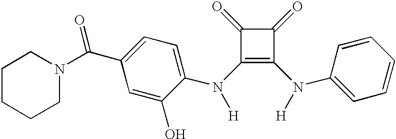 | 1. 60% 2. 392 3. 270-280 |
| 23 | 2 | 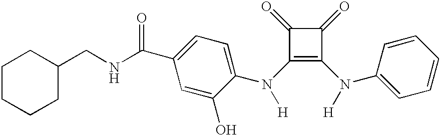 | 1. 47% 2. 420 3. 255-260 |
| 24 | 16 | 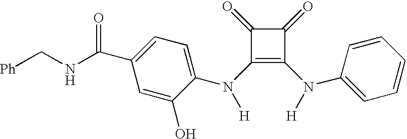 | 1. 53% 2. 414 3. 275-280 |
| 25 | 17 | 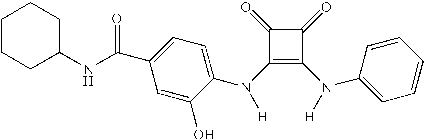 | 1. 62% 2. 406 3. 280-290 |
| 26 | 18 | 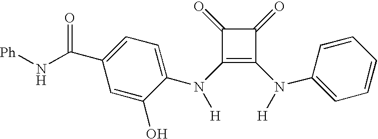 | 1. 77% 2. 400 3. 270-280 |
| 27 | 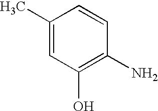 | 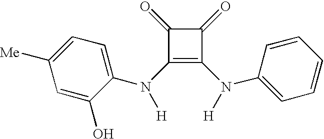 | 1. 61% 2. 295 3. 265-267 |

### EXAMPLE 28

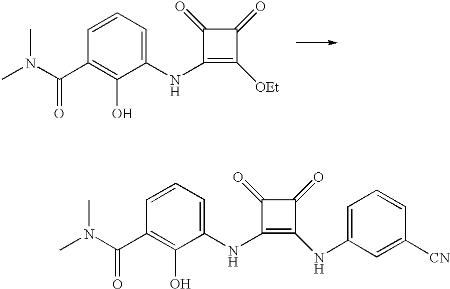

The compound from Preparative Example 31 (100 mg), 3-amino benzonitrile (78 mg), triethylamine (0.23 mL) and absolute ethanol (10 mL) were heated at 80° C. overnight. The reaction mixture was concentrated in vacuo, diluted with 1 N NaOH (aq) and washed with dichloromethane. The aqueous phase was acidified (1 M HCl), extracted with EtOAc, and the organic phase was dried over Na₂SO₄, filtered and concentrated in vacuo. The residue was purified by column chromatography (silica gel, 5% MeOH/CH₂Cl₂ saturated with NH₄OH) to give the product (35 mg, 28%, MH⁺⁼377, mp=135-140° C.).

### EXAMPLES 29-37

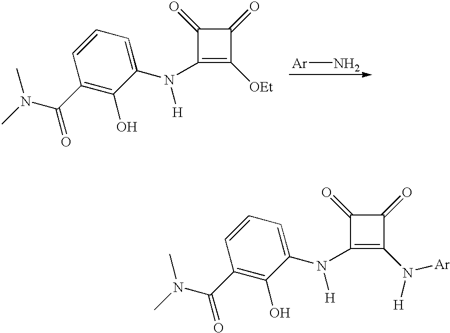

Following the procedure described for Example 28, using the aromatic amines shown below instead of 3-aminobenzonitrile, the Products listed in Table VII below were prepared. In some cases the product precipitated from the solution and could be isolated without further purification.

TABLE VII

| | | | 1. Yield (%) |
|---|---|---|---|
| | | | 2. MH⁺ |
| Example | Aromatic Amine | Product | 3. mp (° C.) |
| 29 | 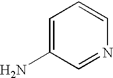 | 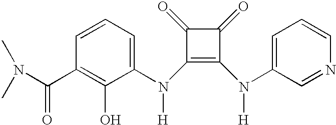 | 1. 45 2. 353 3. 88-93 |
| 30 | 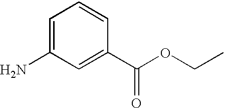 | 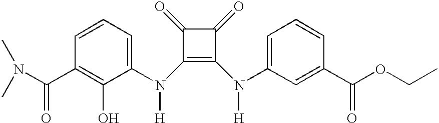 | 1. 25 2. 424 3. 123-128 |
| 31 | 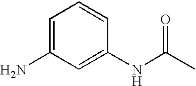 | 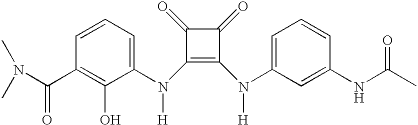 | 1. 40 2. 409 3. 225-230 |
| 34 | 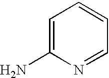 | 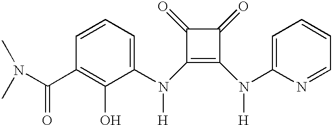 | 1. 13 2. 353 3. 292.6 |
| 36 | 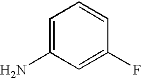 | 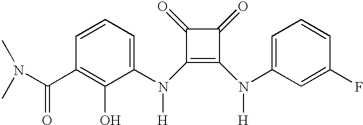 | 1. 75 2. 370 3. 125-130 |
| 37 | 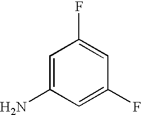 | 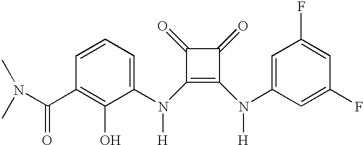 | 1. 12 2. 135-139 3. 388 |

### EXAMPLES 38

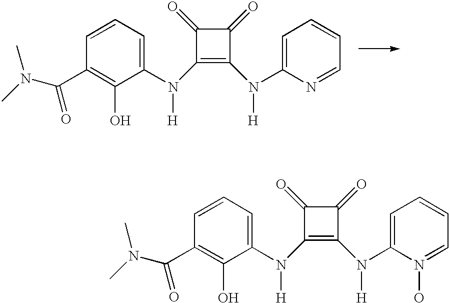

2-aminopyridine is oxidized according to the known procedure (Farmaco 1993, 48, 857-869) to obtain the resulting pyridyl N-oxide which is coupled with the compound from Preparative Example 31 according to the procedure described in Example 28 to give the desired compound.

### EXAMPLE 39

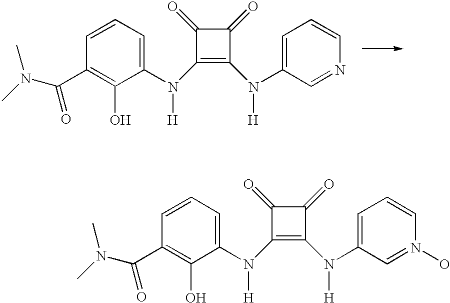

3-aminopyridine is oxidized according to the known procedure (Chem. Lett. 1998, 8, 829-830) to obtain the resulting pyridyl N-oxide which is coupled with the compound from Preparative Example 31 according to the procedure described in Example 28 to give the desired compound.

### EXAMPLE 40

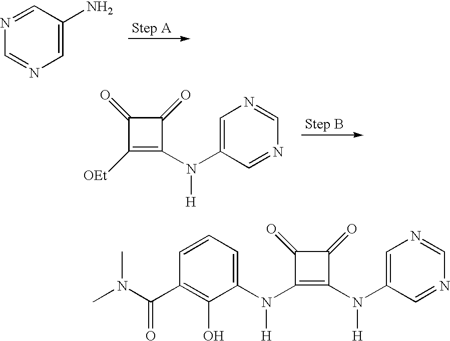

Step A

Following the procedure outlined in Preparative Example 30 using the commercially available 3-aminopyrazine instead of aniline, the ethoxy intermediate is obtained.

Step B

The ethoxy intermediate from Step A above is condensed with the compound from Preparative Example 19 according to the procedure used in Preparative Example 1 to obtain the title compound.

### EXAMPLES 41-43

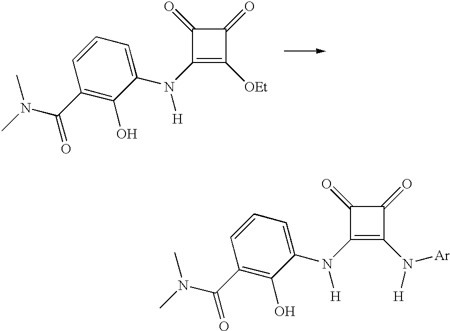

Following the procedure described in Example 40, using the aromatic amines shown below instead of 3-aminopyrazine, the Products listed in Table VIII below can be obtained.

TABLE VIII

| Example | Aromatic Amine | Product |
|---|---|---|
| 41 | 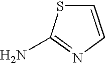 | 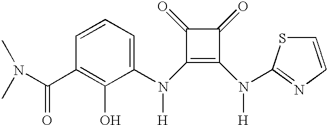 |
| 42 | 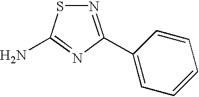 | 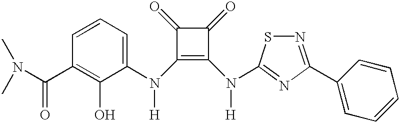 |
| 43 | 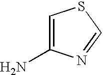 | 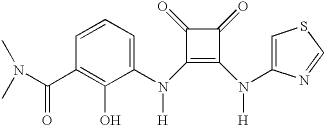 |

### EXAMPLE 44

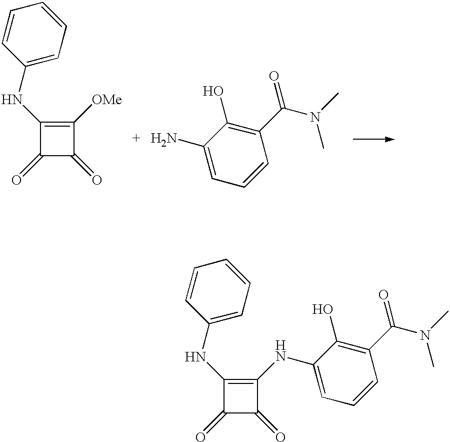

The N,N-dimethylamide from Preparative Example 33 (0.74 g, 4.1 mmol) and the methyl squarate derivative from Preparative Example 66 (0.84 g, 4.1 mmol) were combined in methanol and heated to reflux. The mixture was stirred for 96 hours. After this time, LCMS showed the desired product was present. The reaction was concentrated and product was isolated by HPLC purification (102.6 mg, 7.31%).

¹H NMR (300 MHz, d₆-DMSO)δ 2.95 (s, 6H), 6.94 (m, 2H), 7.09 (m, 1H), 7.39 (m, 2H), 7.51 (d, 2H), 7.74 (dd, 1H).

LCMS: calculated: 351.12, found: 352.0 (M+1)⁺

### Examples 45-82

Following the procedure described for Example 44, the Products listed in Table IX below were prepared using the aniline from the Preparative Example indicated (or the commercially available aniline illustrated) and the alkoxy squarate from the preparative example indicated. The reaction was complete in 16-96 hrs depending on the aniline as determined by TLC.

TABLE IX

| | Aniline and Squarate | | 1. Yield (%) |
|---|---|---|---|
| Example | from Prep Exs. | Product | 2. (M + 1)⁺ |
| 45 | 47 & 66 | 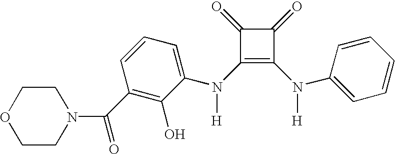 | 1. 32% 2. 394.0 |
| 46 | 45 & 66 | 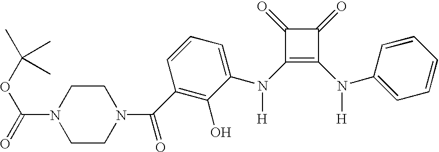 | 1. 4.5% 2. 429.6 |
| 47 | 41 & 66 | 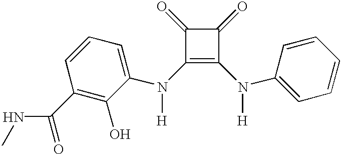 | 1. 0.42% 2. 338.0 |
| 48 | 52 & 66 | 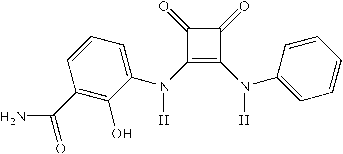 | 1. 7.8 2. 324.0 |
| 49 | 44 & 66 | 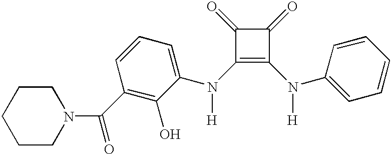 | 1. 6.76% 2. 392.1 |
| 50 | 32 & 66 | 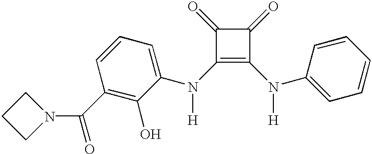 | 1. 10% 2. 364.1 |
| 51 | 53 & 66 | 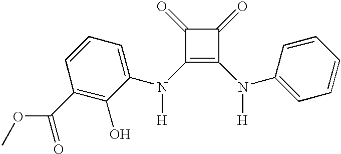 | 1. 3.7% 2. 339.1 |
| 52 | 43 & 66 | 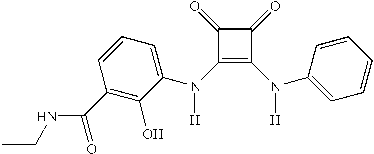 | 1. 0.33% 2. 352.1 |
| 53 | 37 & 66 | 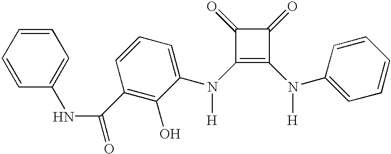 | 1. 5.7% 2. 400.0 |
| 54 | 40 & 66 | 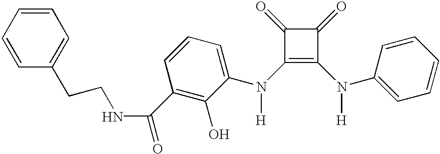 | 1. 11% 2. 428.0 |
| 55 | 34 & 66 | 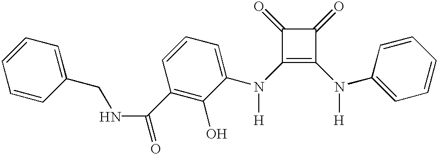 | 1. 1.2% 2. 414.1 |
| 56 | 35 & 66 | 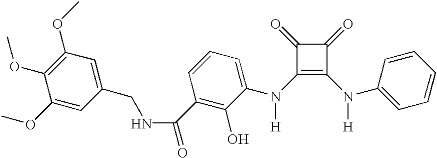 | 1. 5.1% 2. 504.0 |
| 57 | 36 & 66 | 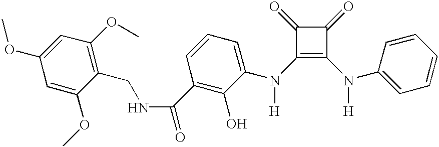 | 1. 6.7 2. 503.8 |
| 58 | 42 & 66 | 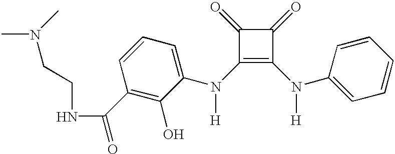 | 1. 3.6% 2. 395.1 |
| 59 | 39 & 66 | 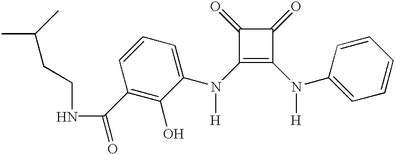 | 1. 9.4% 2. 394.1 |
| 60 | 38 & 66 | 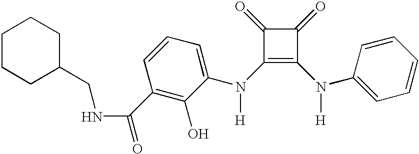 | 1. 0.40% 2. 420.1 |
| 61 | 48 & 66 | 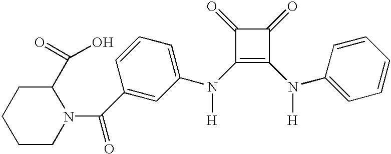 | 1. 10% 2. 420.0 |
| 62 | 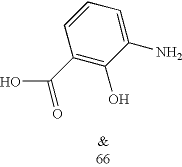 | 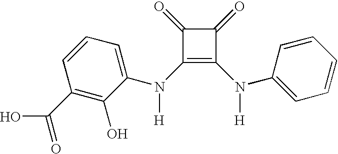 | 1. 24% 2. 295.0 |
| 63 | 33 & 78 | 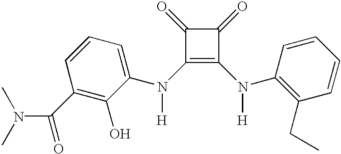 | 1. 53% 2. 380.1 |
| 64 | 33 & 79 | 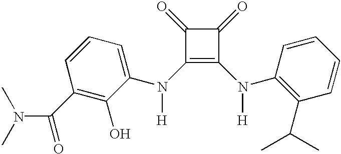 | 1. 16% 2. 394.0 |
| 65 | 33 & 80 | 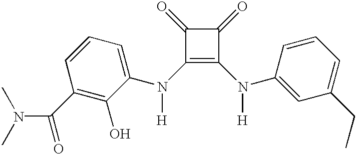 | 1. 43% 2. 380.1 |
| 66 | 33 & 81 | 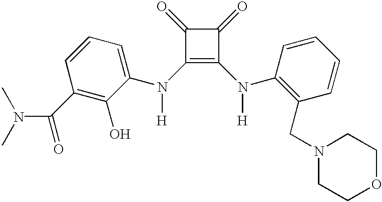 | 1. 44% 2. 451.1 |
| 67 | 33 & 82 | 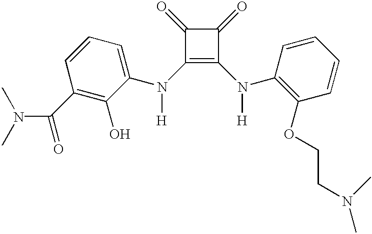 | 1. 42% 2. 439.1 |
| 68 | 33 & 74 | 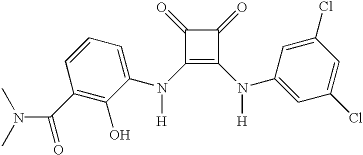 | 1. 45% 2. 420.0 |
| 69 | 33 & 76 | 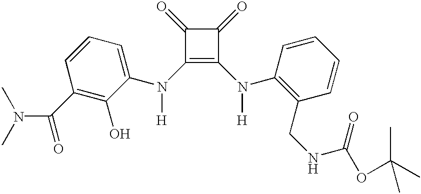 | 1. 32% 2. 481.0 |
| 70 | 33 & 83 | 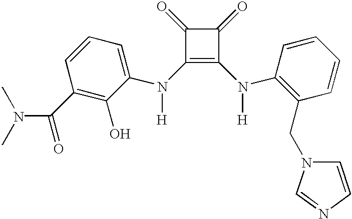 | 1. 20% 2. 432.0 |
| 71 | 33 & 77 | 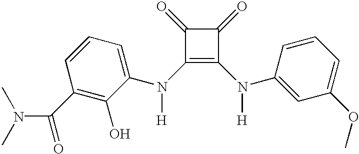 | 1. 30% 2. 382.0 |
| 72 | 33 & 72 | 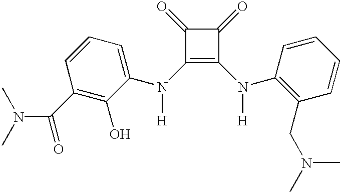 | 1. 15% 2. 409.0 |
| 73 | 33 & 73 | 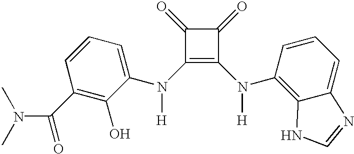 | 1. 57% 2. 359.0 |
| 74 | 33 & 71 | 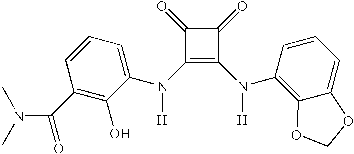 | 1. 25% 2. 396.0 |
| 75 | 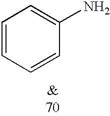 | 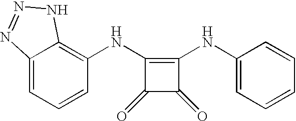 | 1. 39% 2. 306.0 |
| 76 | 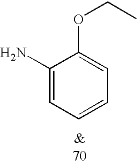 | 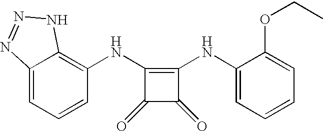 | 1. 34% 2. 350.1 |
| 77 | 58 & 70 | 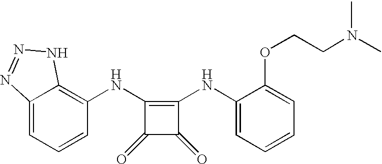 | 1. 75% 2. 393.1 |
| 78 | 63 & 70 | 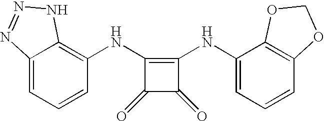 | 1. 26% 2. 350.1 |
| 79 | 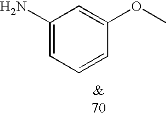 | 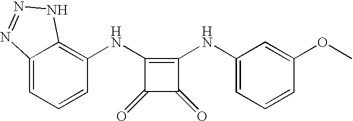 | 1. 26% 2. 336.1 |
| 80 | 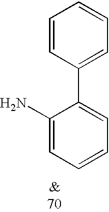 | 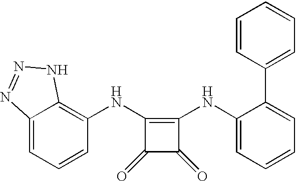 | 1. 23% 2. 382.1 |
| 81 | 61 & 70 | 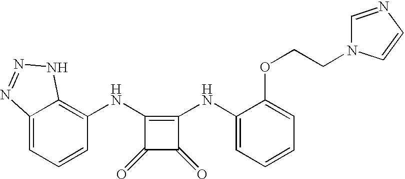 | 1. 60% 2. 416.1 |
| 82 | 59 & 70 | 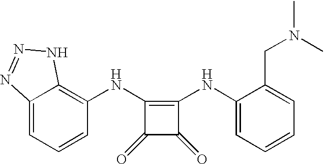 | 1. 59% 2. 363.1 |

### Example 83

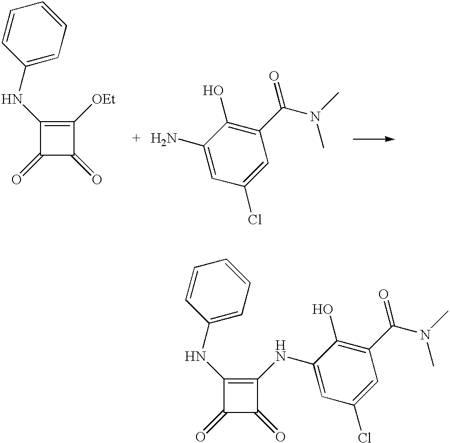

The aniline 314 from Preparative Example 46 (52 mg, 0.25 mmol) and the ethoxy squarate derivative from Preparative Example 67 (50 mg, 0.25 mmol) were combined in ethanol (2 mL) with diisopropylethylamine (0.10 mL) and heated to reflux for 16 hours. The reaction was concentrated and the product was isolated by HPLC purification (7.2 mg, 7.4%).

¹H NMR (300 MHz, d₆-DMSO)δ 3.04 (s, 6H), 7.02 (d, 1H), 7.20 (t, 1H), 7.48 (t, 2H), 7.59 (m, 2H), 8.03 (d, 1H), 9.70 (s, 1H), 10.34 (s, 1H), 10.60 (s, 1H)ppm.

LCMS: calculated: 385.1, found: 386.0 (M+1)⁺

### Examples 84-93

Following the procedure described for Example 83, the Products listed in Table X below were prepared using the amine from the Preparative Example indicated (or the commercially available aniline illustrated) and the ethoxy squarate from the preparative example indicated.

TABLE X

| | Aniline and Squarate | | 1. Yield (%) |
|---|---|---|---|
| Example | from Prep Exs. | Product | 2. (M + 1)⁺ |
| 84 | 33 & 68 | 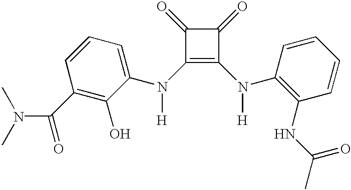 | 1. 22% 2. 409.0 |
| 85 | 33 & 69 | 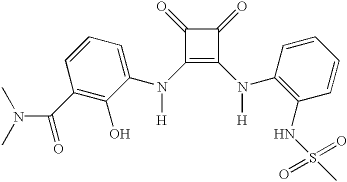 | 1. 14% 2. 445.0 |
| 86 | 34 & 75 | 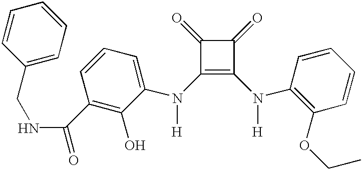 | 1. 24% 2. 458.0 |
| 87 | 49 & 67 | 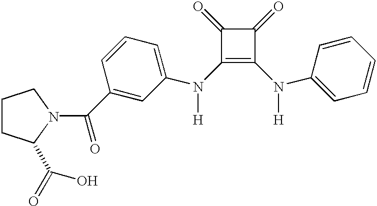 | 1. 33% 2. 406.0 |
| 88 | 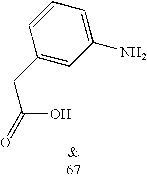 | 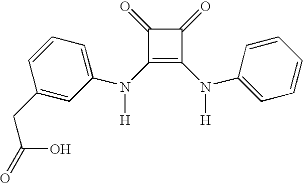 | 1. 55% 2. 323.0 |
| 89 | 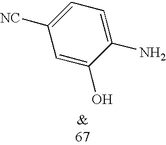 | 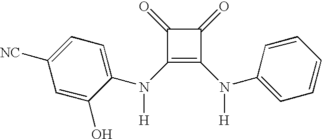 | 1. 21% 2. 306.1 |
| 90 | 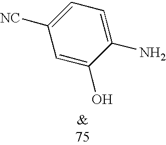 | 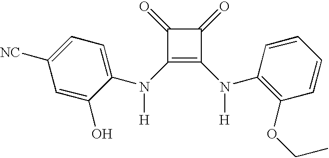 | 1. 52% 2. 350.1 |
| 91 | 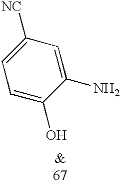 | 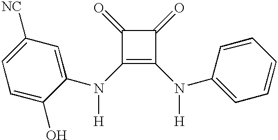 | 1. 2.6% 2. 306.0 |
| 92 | 50 & 67 | 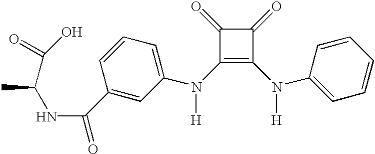 | 1. 30% 2. 380.0 |
| 93 | 51 & 67 | 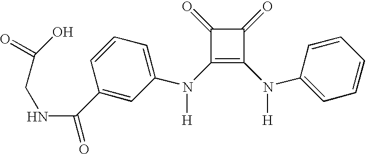 | 1. 38% 2. 366.0 |

### EXAMPLE 94

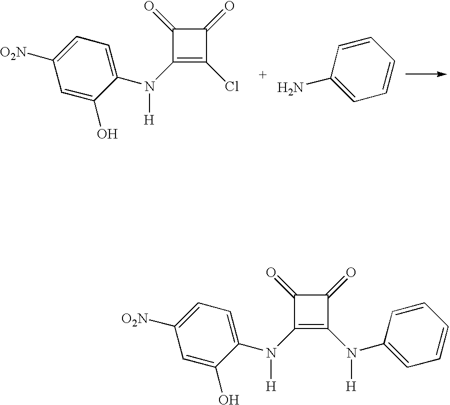

The compound from Preparative Example 90 (50 mg, 0.11 mmol) was dissolved in tetrahydrofuran (2mL). Aniline (0.017 mL, 0.19 mmol) was added, and the mixture was stirred for 2hr. The solvent was evaporated, and the residue was taken up in acetonitrile. The desired product (30 mg, 49% yield), an insoluble powder, was recovered by filtration.

¹H NMR (300 MHz, d₆-DMSO)δ 7.18 (m, 1H), 7.35 (m, 1H), 7.48 (m, 2H), 7.54 (m, 1H), 7.83 (m, 2H), 8.13 (d, 1H), 9.95 (s, 1H), 10.86 (s, 1H), 11.50 (s, 1H)ppm.

Mass Spec.: calculated 325.0, found 326.1 (M+1)⁺

### Examples 95-105

Following the procedure described for Example 94, the Products listed in Table XI below were prepared using the aniline from the Preparative Example indicated (or the commercially available aniline illustrated) and the chloride from the preparative example indicated.

TABLE XI

| | Aniline and | | |
|---|---|---|---|
| | Chloride from | | 1. Yield (%) |
| Example | Prep Exs. | Product | 2. (M + 1)⁺ |
| 95 | 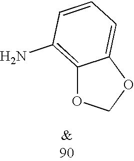 | 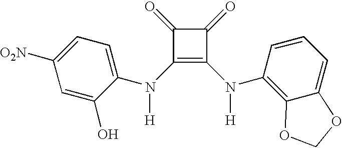 | 1. 27% 2. 370.1 |
| 96 | 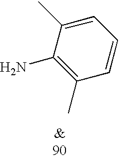 | 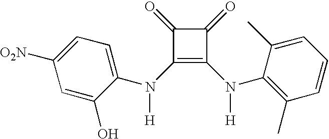 | 1. 21% 2. 354.1 |
| 97 | 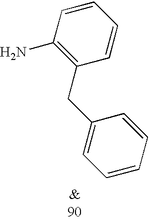 | 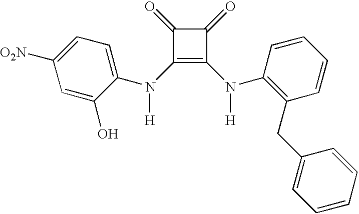 | 1. 20% 2. 416.0 |
| 98 | 65 & 90 | 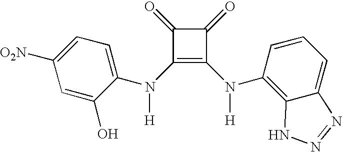 | 1. 5.0% 2. 367.1 |
| 99 | 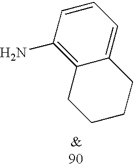 | 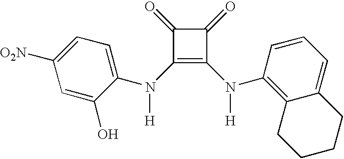 | 1. 21% 2. 354.1 |
| 100 | 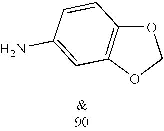 | 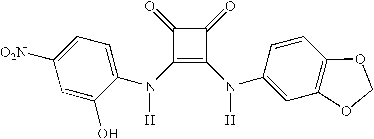 | 1. 6.8% 2. 370.1 |
| 101 | 89 & 90 | 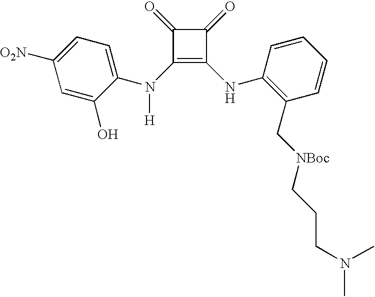 | 1. 31% 2. 540.0 |
| 102 | 42 & 90 | 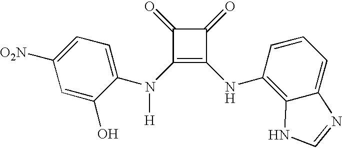 | 1. 40% 2. 366.1 |
| 104 | 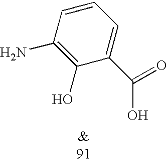 | 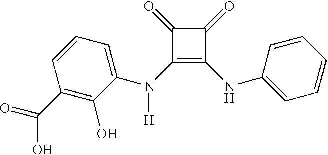 | 1. 22% 2. 324.9 |
| 105 | 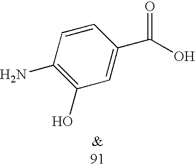 | 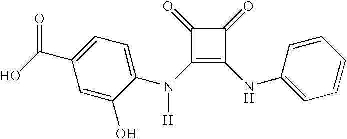 | 1. 10% 2. 325.0 |
| 106 | 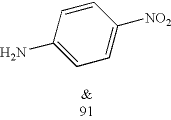 | 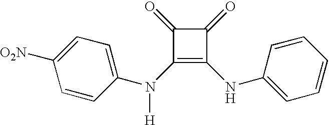 | 1. 21% 2. 310.2 |

### EXAMPLE 107

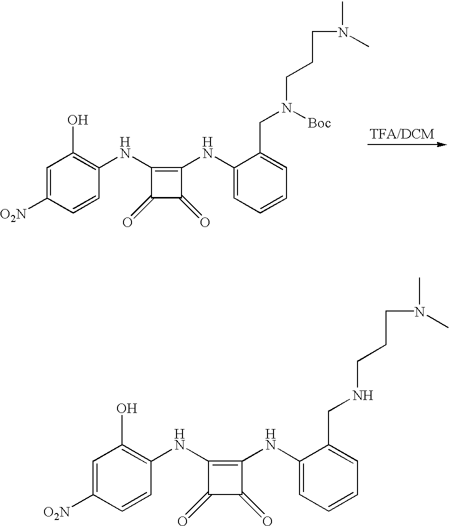

The Boc-protected compound of Example 101 (14.5 mg, 0.027 mol) was stirred in TFA/DCM (5 mL/5 mL) for 2 h. Simple concentration gave the product (11.2 mg, 95%).

¹H NMR (300 MHz, d₆-DMSO)δ 2.08 (t, 2H), 2.82 (s, 6H), 3.18 (m, 4H), 4.40 (s, 2H), 7.43 (m, 2H), 7.58 (d, 1H), 7.65 (d, 1H), 7.80 (s, 1H), 7.90 (d, 1H), 8.18 (d, 1H), 9.18 (1H), 9.80 (m, 1H), 10.43 (s, 1H), 11.62 (s, 1H)ppm.

LCMS(MH⁺): 439.19, found 439.8.

### EXAMPLE 108

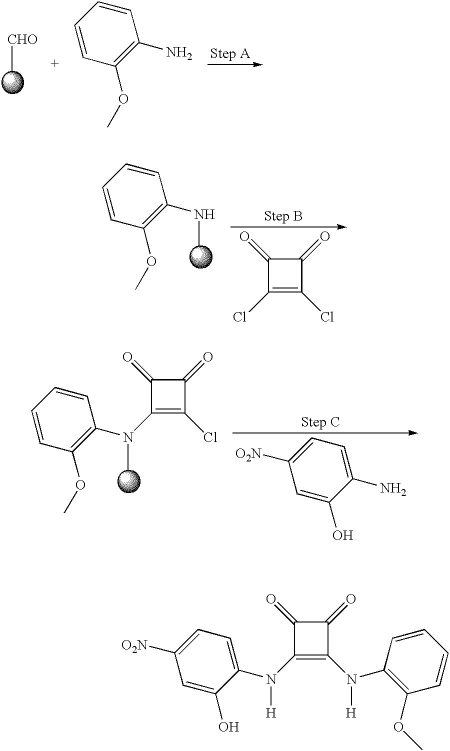

General Procedure for Resin Preparation

Resin Double-Loading:

Argogel (NH2) resin (10 g, 160u, 0.4 mmol/g) was suspended in dicloromethane (100 mL) in a large peptide vessel. Bis-(Fmoc)-lysine (7.09 g, 12 mmol) and 1-hydroxybenzotriazole hydrate (1.62 g, 12 mmol) were dissolved in dichoromethane (100 mL) with N,N-dimethylformamide (12 mL) and added to the vessel. The vessel was shaken for 10 min. 1,3-Diisopropylcarbodiimide (3.76 mL, 24 mmol) was added to the vessel with frequent venting during the first 15 min of shaking. The mixture was shaken for 16 hr. The resin was filtered and washed three times each with dichloromethane, methanol, and dichloromethane. The resin was dried under vacuum.

Acid-Cleavable Linker Attachment:

The double-loaded resin (0.9 g) was placed in a small peptide vessel with a solution of 20% piperidine in DMF. The mixture was shaken for 2 hr then filtered. The resin was filtered and washed three times each with N,N-dimethylformamide, methanol, and dichloromethane. The resin was suspended in a solution of 4-(4-formyl-3′-methoxy)-phenoxybutyric acid (0.463 g, 2 mmol) and 1-hydroxybenzotriazole hydrate (0.262 g, 2 mmol) in dichloromethane (10 mL). The mixture was shaken for 10 min, then 1,3-diisopropylcarbodiimide was added with frequent venting during the first 15 min. The mixture was shaken for 16 hr. The resin was filtered and washed three times each with dichloromethane, methanol, and dichloromethane. The resin was dried under vacuum.

Step A

The prepared resin (1 g) was suspended with sodium triacetoxyborohydride (1.1 g, 5 mmol) and dichloroethane (10 mL) in a small peptide vessel. o-Anisidine (0.564 mL, 5 mmol) was added, and the mixture was shaken for 16 hr. The resin was filtered and washed successively two times each with methanol, dichloromethane, methanol, and dichloromethane.

Step B

Squaryl chloride (0.690 g, 4.6 mmol) was dissolved in tetrahydrofuran (10 mL) and added to resin from Step A. The mixture was shaken overnight then washed successively two times each with dichloromethane, acetonitrile, and dichloromethane.

Step C

Resin from Step B (0.25 g) was suspended with 2-amino-5-nitrophenol (0.308 g, 2 mmol) and N,N-diisopropylethylamine (0.35 mL, 2 mmol) in tetrahydrofuran (4 mL). The mixture was shaken for 16 hr. The resin was filtered and washed three times each with dichloromethane, methanol, and dicloromethane. For cleavage, the resin was suspended in 90% trifluoroacetic acid/dicloromethane with stirring for 6 hr. The resin was filtered, washed with acetonitrile and discarded. The filtrate and washes were concentrated to give the desired, pure product (11.6 mg, 26% yield).

¹H NMR (300 MHz, d₆-DMSO)δ 4.01 (s, 3H), 7.08 (m, 1H), 7.22 (m, 2H), 7.62 (d, 1H), 7.81 (s, 1H), 7.88 (dd, 1H), 8.09 (d, 1H), 10.33 (s, 1H), 10.42 (s, 1H), 11.38 (s, 1H)ppm.

Mass Spec.: calculated 355.1, found 356.0 (M+1)⁺

### PREPARATIVE EXAMPLES 109-120

Following the procedure described for Example 108, the Products listed in Table XII below were prepared using the commercially available Step A aniline or amine illustrated and the Step C aniline from the Preparative Example indicated (or the commercially available aniline illustrated). (Yields for small scale preparations, <50 mg resin, were not accurate and are indicated in the table as “NA”.)

TABLE XII

| | Step A aniline or | | |
|---|---|---|---|
| | amine/Step C | | 1. Yield (%) |
| Example | aniline | Product | 2. (M+1)* |
| 109 | 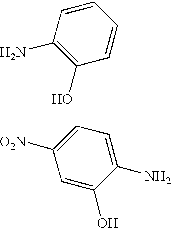 | 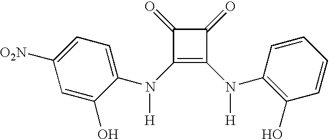 | 1. 32% 2. 342.0 |
| 110 | 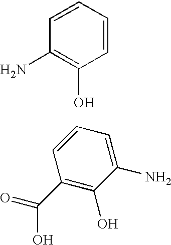 | 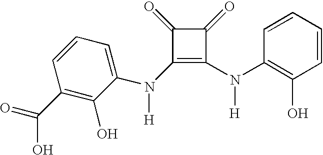 | 1. NA 2. 340.9 |
| 111 | 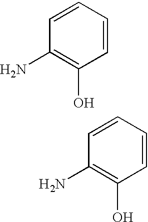 | 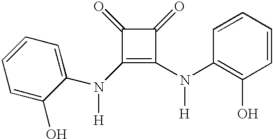 | 1. NA 2. 297.0 |
| 112 | 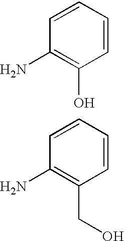 | 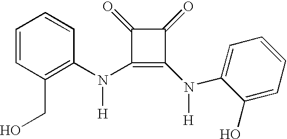 | 1. NA 2. 310.9 |
| 113 | 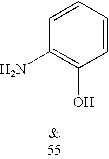 | 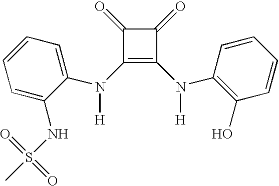 | 1. NA 2. 373.9 |
| 114 | 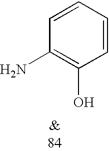 | 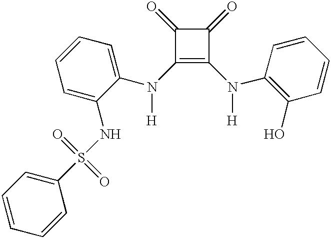 | 1. NA 2. 435.9 |
| 115 | 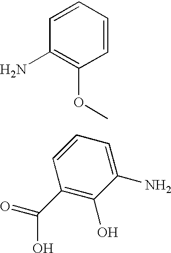 | 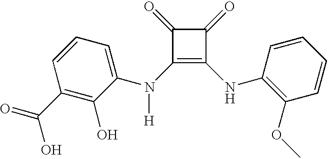 | 1. NA 2. 354.9 |
| 116 | 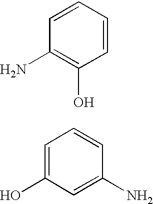 | 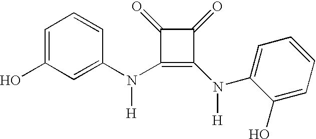 | 1. NA 2. 297.1 |
| 117 | 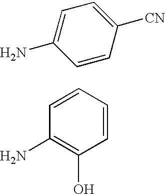 | 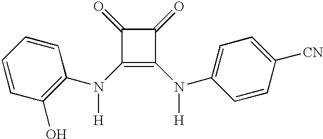 | 1. NA 2. 306.1 |
| 118 | 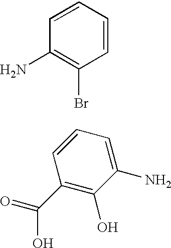 | 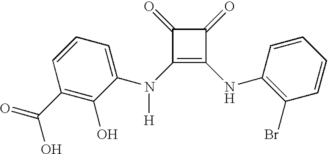 | 1. NA 2. 402.8 |
| 119 | 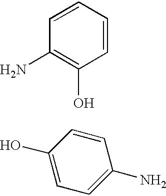 | 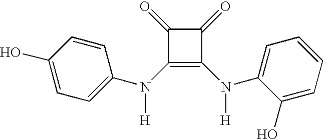 | 1. NA 2. 297.1 |
| 120 | 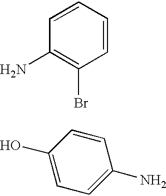 | 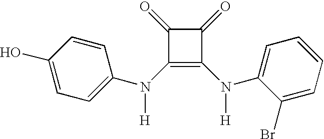 | 1. NA 2. 361.0 |

### EXAMPLE 123

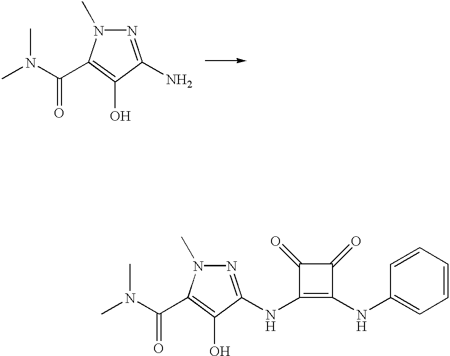

The compound from Preparative Example 26 is reacted with the compound from Preparative Example 30 according to the procedure described in Example 1 to obtain the product shown.

### EXAMPLE 124

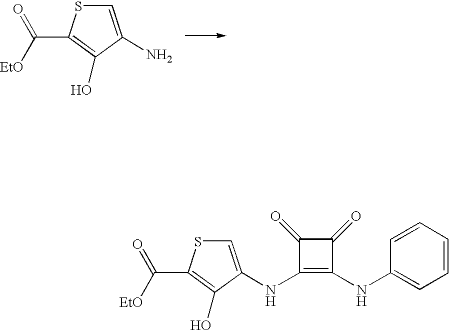

The compound from Preparative Example 27 is reacted with the compound from Preparative Example 30 according to the procedure described in Example 1 to obtain the product shown.

### EXAMPLE 125

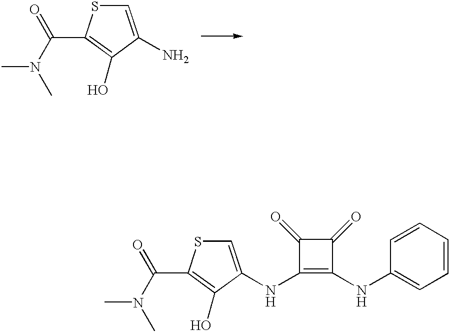

The compound from Preparative Example 28 Step B or Preparative Example 29 Step E is reacted with the compound from Preparative Example 30 according to the procedure described in Example 1 to obtain the product shown.

What is claimed:

1. A compound of the formula 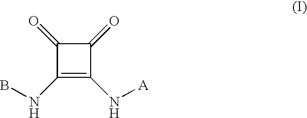
a prodrug thereof, or a pharmaceutically acceptable salt, solvate or isomer of said compound or of said prodrug;
wherein
A is an unsubstituted or substituted aryl or unsubstituted or substituted heteroaryl group;
B is 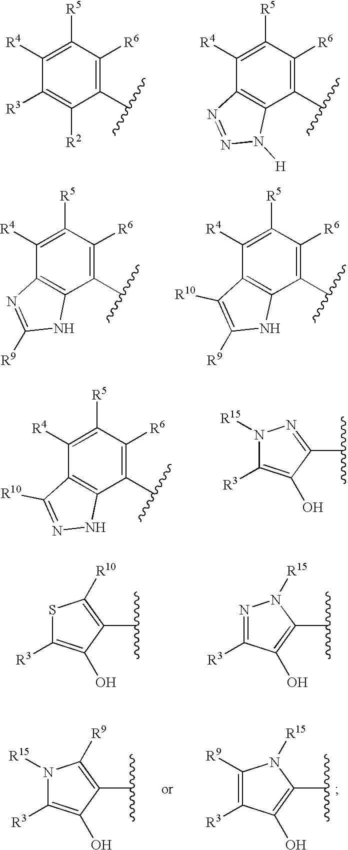
R² is hydrogen, OH, C(O)OH, SH, SO₂NR⁷R⁸, NHC(O)R⁷, NHSO₂NR⁷R⁸, NHSO₂R⁷, C(O)NR⁷R⁸, C(O)N R⁷OR⁸, OR¹³ or an unsubstituted or substituted heterocyclic acidic functional group;
R³ and R⁴ are the same or different and are independently hydrogen, halogen, alkoxy, OH, CF₃, OCF₃, NO₂, C(O)R⁷N C(O)OR⁷, C(O)NR⁷R⁸, SO(H)NR⁷R⁸, SO₍ₜ₎R⁷, C(O)NR⁷OR⁸, 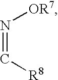
cyano, unsubstituted or substituted alkyl, unsubstituted or substituted aryl or unsubstituted or substituted heteroaryl;
R⁵ and R5 are the same or different and are independently hydrogen, halogen, alkyl, alkoxy, CF₃, OCF₃, NO₂, C(O)R⁷, C(O)OR⁷, C(O)NR⁷R⁸, SO(T)NR⁷R⁸, C(O)NR⁷OR⁸, cyano, or an unsubstituted or substituted aryl or an unsubstituted or substituted heteroaryl group;
R⁷ and R⁸ are the same or different and are independently hydrogen, unsubstituted or substituted alkyl, unsubstituted or substituted aryl, unsubstituted or substituted alkylaryl, unsubstituted or substituted arylalkyl, unsubstituted or substituted cycloalkyl, carboxyalkyl, aminoalkyl, unsubstituted or substituted heteroaryl, unsubstituted or substituted heteroarylalkyl or unsubstituted or substituted heteroalkylaryl, or
R⁷, R⁸ and N in said NR⁷R⁸ and NR⁷OR⁸ can jointly form a 3 to 7 membered ring, said ring may further contain 1 to 3 additional heteroatoms on said ring as ring atoms, and said ring may be unsubstituted or substituted with one or more moieties which are the same or different, each moiety being independently selected from hydroxy, cyano, carboxyl, hydroxyalkyl, alkoxy, COR⁷R⁸ or aminoalkyl;
R⁹ and R¹⁰ are the same or different and are independently hydrogen, halogen, CF₃, OCF₃, NR⁷R⁸, NR⁷C(O)NR⁷R⁸, OH, C(O)OR⁷, SH, SO₍ₜ₎NR⁷R⁸, SO₂R⁷, NHC(O)R⁷, NHSO₂NR⁷R⁸, NHSO₂R⁷, C(O)NR⁷R⁸, C(O)NR⁷OR⁸, OR¹³ or an unsubstituted or substituted heterocyclic acidic functional group;
R¹³ is COR⁷;
R¹⁵ is hydrogen, OR¹³, or an unsubstituted or substituted aryl group, an unsubstituted or substituted heteroaryl group, an unsubstituted or substituted arylalkyl group, an unsubstituted or substituted cycloalkyl group or an unsubstituted or substituted alkyl group; and
t is 1 or 2.

2. The compound according to claim 1
a prodrug thereof, or a pharmaceutically acceptable salt, solvate or isomer of said compound or of said prodrug;
wherein
A is 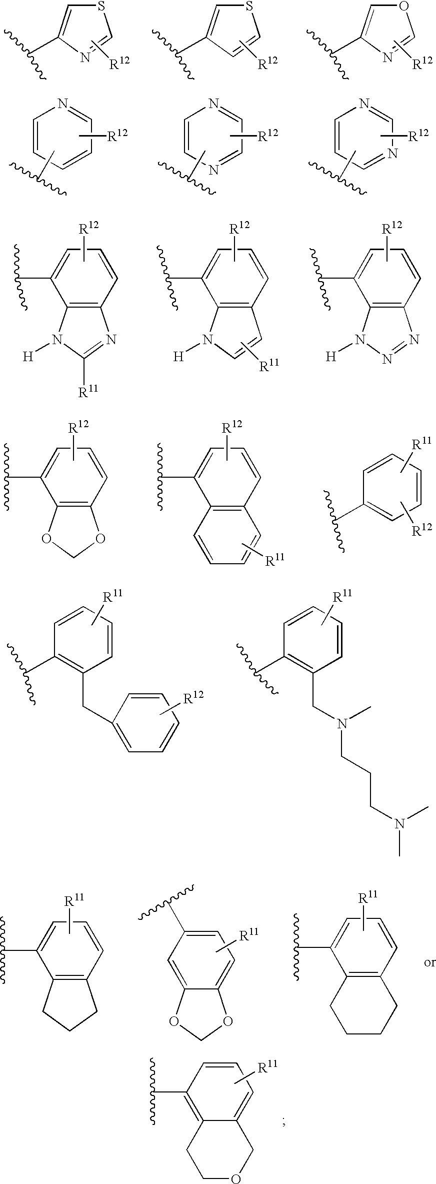
R¹¹ and R¹² are the same or different and are independently H, OH, halogen, cyano, CF₃, CF₃O, NR⁷R⁸, NR⁷C(O)NR⁷R⁸, C(O)NR⁷R⁸, CO₂R⁷, OR⁷, SO(t) NR⁷R⁸, NR⁷SO₍ₜ₎R⁸, COR⁷, and substituted or unsubstituted aryl, substituted or unsubstituted alkyl, substituted or unsubstituted arylalkyl, substituted or unsubstituted heteroaryl, substituted or unsubstituted aryloxy, substituted or unsubstituted heteroarylalkyl, substituted or unsubstituted heterocyclylalkyl, substituted or unsubstituted hydroxyalkyl, substituted or unsubstituted alkoxy, alkylaminoCOOalkyl, aminoalkoxy, alkoxyaminoalkyl or substituted or unsubstituted aminoalkyl.

3. The compound according to claim 1
a prodrug thereof, or a pharmaceutically acceptable salt, solvate or isomer of said compound or of said prodrug;
wherein
R² is hydrogen, OH, NHC(O)R⁷ or NHSO₂R⁷;
R³ is SO₂NR⁷R⁸, C(O)NR⁷R⁸, SO₂R⁷, NO₂ or cyano;
R⁴ is hydrogen, NO₂, CF₃orcyano,
R⁵ is hydrogen, halogen, NO₂, cyano or CF₃; and
R⁶ is hydrogen or CF₃.

4. The compound according to claim 2
a prodrug thereof, or a pharmaceutically acceptable salt, solvate or isomer of said compound or of said prodrug;
wherein
A is

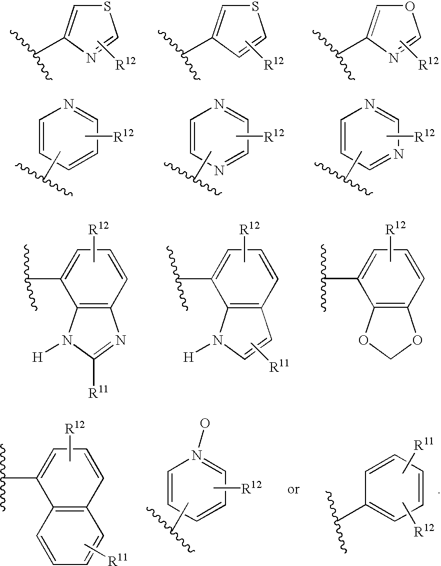

5. The compound according to claim 2
a prodrug thereof, or a pharmaceutically acceptable salt, solvate or isomer of said compound or of said prodrug;
wherein
R² is hydrogen, OH, NHC(O)R⁷ or NHSO₂R⁷;
R³ is SO₂NR⁷R⁸, C(O)NR⁷R⁸, SO₂R⁷, NO₂ or cyano;
R⁴ is hydrogen, NO₂, CF₃orcyano;
R⁵ is hydrogen, halogen, cyano, NO₂ or CF₃; and
R⁶ is hydrogen or CF₃.

6. The compound according to claim 4
a prodrug thereof, or a pharmaceutically acceptable salt, solvate or isomer of said compound or of said prodrug;
wherein
R² is hydrogen, OH, NHC(O)R⁷ or NHSO₂R⁷;
R³ is SO₂NR⁷R⁸, C(O)NR⁷R⁸, SO₂R⁷, NO₂ or cyano;
R⁴ is hydrogen, NO₂, CF₃ or cyano;
R⁵ is hydrogen, halogen or CF₃; and
R⁶ is hydrogen or CF₃.

7. The compound according to claim 3
a prodrug thereof, or a pharmaceutically acceptable salt, solvate or isomer of said compound or of said prodrug;
wherein
R² is OH or NHSO₂R⁷;
R³ is C(O)NR⁷R⁸, NO₂ or cyano;
R⁴ is hydrogen, NO₂ or cyano;
R⁵ is hydrogen, Cl or CF₃; and
R⁶ is hydrogen or CF₃.

8. The compound according to claim 7
a prodrug thereof, or a pharmaceutically acceptable salt, solvate or isomer of said compound or of said prodrug;
wherein
R² is OH;
R³ is C(O)NR⁷R⁸;
R⁴ is hydrogen;
R⁵ is hydrogen, Cl or CF₃; and
R⁶ is hydrogen.

9. The compound according to claim 5
a prodrug thereof, or a pharmaceutically acceptable salt, solvate or isomer of said compound or of said prodrug;
wherein
R² is OH or NHSO₂R⁷;
R³ is C(O)NR⁷R⁸, NO₂ or cyano;
R⁴ is hydrogen, NO₂orcyano;
R⁵ is hydrogen, Cl or CF₃; and
R⁶ is hydrogen or CF₃.

10. The compound according to claim 6
a prodrug thereof, or a pharmaceutically acceptable salt, solvate or isomer of said compound or of said prodrug;
wherein
R² is OH or NHSO₂R⁷;
R³ is C(O)NR⁷R⁸, NO₂ or cyano;
R⁴ is hydrogen, NO₂orcyano;
R⁵ is hydrogen, Cl or CF₃; and
R⁶ is hydrogen or CF₃.

11. The compound according to claim 9
a prodrug thereof, or a pharmaceutically acceptable salt, solvate or isomer of said compound or of said prodrug;
wherein
R² is OH;
R³ is C(O)NR⁷R⁸;
R⁴ is hydrogen;
R⁵ is hydrogen, Cl or CF₃; and
R⁶ is hydrogen.

12. The compound according to claim 10
a prodrug thereof, or a pharmaceutically acceptable salt, solvate or isomer of said compound or of said prodrug;
wherein
R² is OH;
R³ is C(O)NR⁷R⁸;
R⁴ is hydrogen;
R⁵ is hydrogen, Cl or CF₃; and
R⁶ is hydrogen.

13. A compound according to claim 1
a prodrug thereof, or a pharmaceutically acceptable salt, solvate or isomer of said compound or of said prodrug;
wherein A and B are as shown in the following table:

| Ex. | A | B |
|---|---|---|
| 20 | 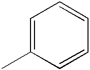 | 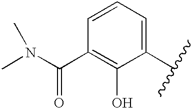 |
| 36 | 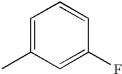 | 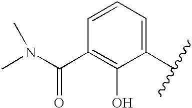 |
| 37 | 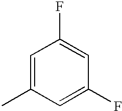 | 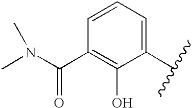 |
| 45 | 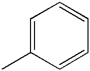 | 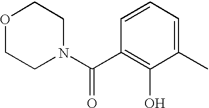 |
| 49 | 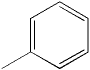 | 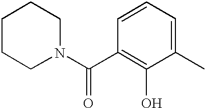 |
| 50 | 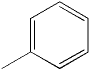 | 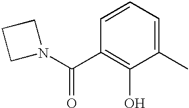 |
| 63 | 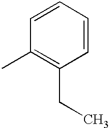 | 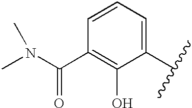 |
| 64 | 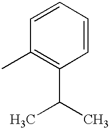 | 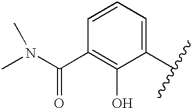 |
| 65 | 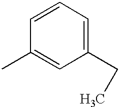 | 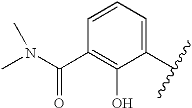 |
| 66 | 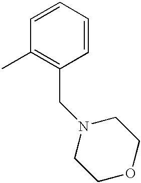 | 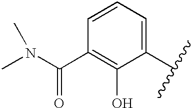 |
| 71 | 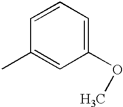 | 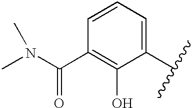 |
| 74 | 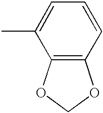 | 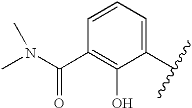 |
| 89 | 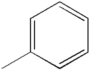 | 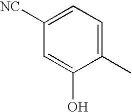 |
| 90 | 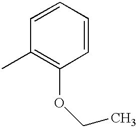 | 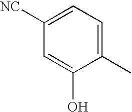 |
| 96 | 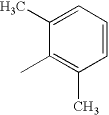 | 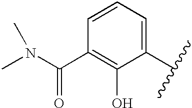 |
| | 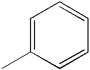 | 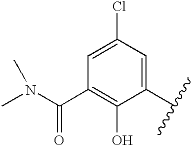 |
| | 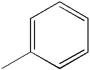 | 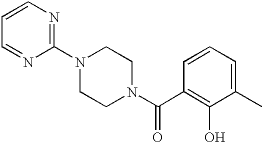 |
| | 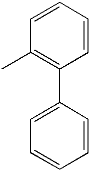 | 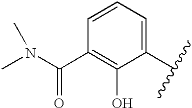 |

14. The compound according to claim 13 of the formula 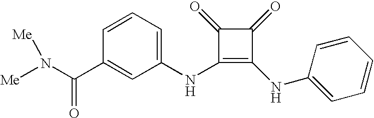
a prodrug thereof, or a pharmaceutically acceptable salt, solvate or isomer of said compound or of said prodrug.

15. The compound according to claim 13 of the formula 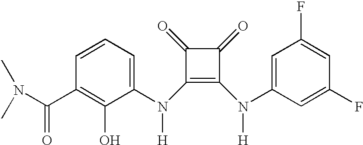
a prodrug thereof, or a pharmaceutically acceptable salt, solvate or isomer of said compound or of said prodrug.

16. The compound according to claim 13 of the formula 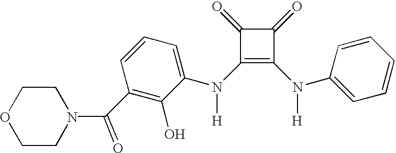
a prodrug thereof, or a pharmaceutically acceptable salt, solvate or isomer of said compound or of said prodrug.

17. The compound according to claim 13 of the formula 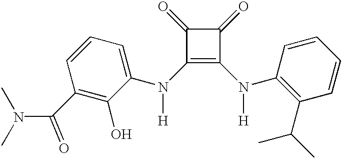
a prodrug thereof, or a pharmaceutically acceptable salt, solvate or isomer of said compound or of said prodrug.

18. The compound according to claim 13 of the formula 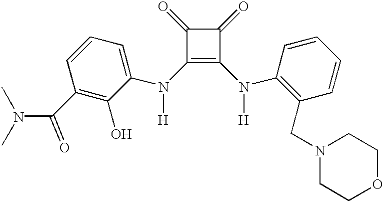
a prodrug thereof, or a pharmaceutically acceptable salt, solvate or isomer of said compound or of said prodrug.

19. The compound according to claim 13 of the formula 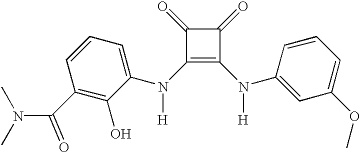
a prodrug thereof, or a pharmaceutically acceptable salt, solvate or isomer of said compound or of said prodrug.

20. The compound according to claim 13 of the formula 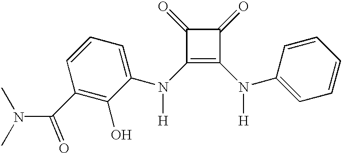
a prodrug thereof, or a pharmaceutically acceptable salt, solvate or isomer of said compound or of said prodrug.

21. The compound according to claim 13 of the formula 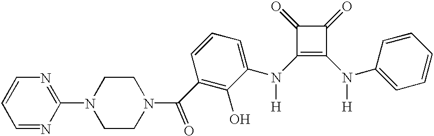
a prodrug thereof, or a pharmaceutically acceptable salt, solvate or isomer of said compound or of said prodrug.

22. A pharmaceutical composition comprising the compound of claim 1, a prodrug thereof, or a pharmaceutically acceptable salt, solvate or isomer of said compound or of said prodrug and a pharmaceutically acceptable carrier therefor.

23. A method of treating a chemokine-mediated disease wherein the chemokine binds to a CXCR2 and/or CXCR1 receptor in a mammal, which comprises administering to a patient in need thereof a therapeutically effective amount of the compound of claim 1, or a pharmaceutically acceptable salt, solvate or isomer of said compound or of said prodrug.

24. A method of treating a chemokine-mediated disease wherein the chemokine binds to a CXC receptor in a mammal, which comprises administering to a patient in need thereof a therapeutically effective amount of the compound of claim 1, or a pharmaceutically acceptable salt, solvate or isomer of said compound or of said prodrug.

25. The method of claim 23 wherein the chemokine mediated disease is selected from the group consisting of psoriasis, atopic dermatitis, asthma, chronic obstructive pulmonary disease, adult respiratory disease, arthritis, inflammatory bowel disease, Crohn's disease, ulcerative colitis, septic shock, endotoxic shock, gram negative sepsis, toxic shock syndrome, stroke, cardiac and renal reperfusion injury, glomerulonephritis or thrombosis, Alzheimer's disease, graft vs. host reaction, allograft rejections, malaria, acute respiratory distress syndrome, delayted type hypersensitivity reaction, atherosclerosis and cerebral and cardiac ischemia.

26. A method of treating cancer, which comprises administering to a patient in need thereof, a therapeutically effective amount of the compound of claim 1, or a pharmaceutically acceptable salt, solvate or isomer of said compound or of said prodrug.

27. The method of claim 26 which further comprises administering to the patient at least one anti-cancer agent and/or radiation therapy.

28. The method of claim 27, wherein the anti-cancer agent is selected from the group consisting of alkylating agents, antimetabolites, natural products and their derivatives, hormones, anti-hormones, anti-angiogenic agents, steroids and synthetics

29. A method of inhibiting angiogenesis which comprises administering to a patient in need thereof an anti-angiogenic amount of the compound of claim 1, or a pharmaceutically acceptable salt, solvate or isomer of said compound or of said prodrug.

30. The method of claim 29 which further comprises administering to the patient at least one known anti-angiogenic agent.

31. The method of claim 30 wherein the known anti-angiogenic agent is selected from the group consisting of Marimastat, AG3340, Col-3, Neovastat, BMS-275291, Thalidomide, Squalamine, Endostatin, SU-5416, SU-6668, Interferon-alpha, Anti-VEGF antibody, EMD121974, CAI, Interleukin-12, IM862, Platelet Factor4, Vitaxin, Angiostatin, Suramin, TNP-470, PTK-787, ZD-6474, ZD-101, Bay 129566, CGS27023A, VEGF receptor kinase inhibitors, taxotere and Taxol.

32. A method of treating a disease selected from the group consisting of gingivitis, respiratory viruses, herpes viruses, hepatitis viruses, HIV, kaposi's sarcoma associated virus and atherosclerosis which comprises administering to a patient in need thereof a therapeutically effective amount of the compound of claim 1, or a pharmaceutically acceptable salt, solvate or isomer of said compound or of said prodrug.

33. The method of claim 23 wherein the chemokine mediated disease is an angiogenic ocular disease.

34. The method of claim 33 wherein the angiogenic ocular disease is selected from the group consisting of ocular inflammation, retinopathy of prematurity, diabetic retinopathy, macular degeneration with the wet type preferred and comeal neovascularization.

35. The method of claim 26 wherein the cancerous tumor type is melanoma, gastric carcinoma or non-small cell lung carcinoma.

36. The method of claim 35 which further comprises administering to the patient at least one anti-cancer agent and/or radiation therapy.

37. The method of claim 36, wherein the anti-cancer agent is selected from the group consisting of alkylating agents, antimetabolites, natural products and their derivatives, hormones, anti-hormones, anti-angiogenic agents, steroids and synthetics

38. The method of claim 37 wherein the anti-angiogenic agent is selected form the group consisting of Marimastat, AG3340, Col-3, Neovastat, BMS-275291, Thalidomide, Squalamine, Endostatin, SU-5416, SU-6668, Interferon-alpha, Anti-VEGF antibody, EMD121974, CAI, Interleukin-12, IM862, Platelet Factor-4, Vitaxin, Angiostatin, Suramin, TNP-470, PTK-787, ZD-6474, ZD-101, Bay 129566, CGS27023A, VEGF receptor kinase inhibitors, taxotere and Taxol.
